(19) United States
(12) Patent Application Publication
(10) Pub. No.: US 2006/0031949 A1
(43) Pub. Date: Feb. 9, 2006

(54) Inducible expression systems for modulating the expression of target genes in eukaryotic cells and non-human animals

(76) Inventor: Yu Shen, Gurnee, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/198,566

(22) Filed: Aug. 5, 2005

Related U.S. Application Data
(63) Continuation-in-part of application No. 11/049,915, filed on Feb. 3, 2005.
Application No. 11/049,915 is a continuation-in-part of application No. 10/913,245, filed on Aug. 6, 2004.

(51) Int. Cl. A01K 67/033; C07H 21/04

(52) U.S. Cl. 800/8; 536/23.1; 435/320.1; 435/325

(57) ABSTRACT

The present invention relates to inducible expression systems and to compositions and methods for modulating the expression of at least one target gene in an eukaryotic cell and non-human animal.

The present application is a continuation-in-part of pending U.S. application Ser. No. 11/049,915, filed on Feb. 3, 2005 which is a continuation-in-part of U.S. application Ser. No. 10/913,245, filed on Aug. 6, 2004, both of which are hereby incorporated in their entirety by reference.

### FIELD OF THE INVENTION

The present invention relates generally to the field of molecular biology. More specifically, the present invention relates to inducible expression systems for use in modulating the expression of target genes in eukaryotic cells and non-human animals.

### BACKGROUND OF THE INVENTION

RNA interference (RNAi) is a process for silencing gene expression using double-stranded RNA. The RNAi mechanism is conserved in plants, invertebrates and vertebrates. Because of its simplicity and specificity, RNAi is becoming the method of choice for studying gene function in a variety of model organisms (See, for example, Hannon, G. J., Nature, 418:244-251 (2002), Paddison, et al., Cancer Cell, 2:17-23 (2002), Sharp, P. A., Genes Dev., 15:485-490 (2001), Tuschl, T., Chembiochem., 2:239-245 (2001) and Zamore, P. D., Nat. Struct. Biol., 8:746-750 (2001)). Although chemically synthesized small interfering RNA (siRNA) can effectively silence genes of interest when transfected into cells, the use of siRNA has been limited to short term experiments because siRNA is degraded over time or diluted after cell division. To overcome this limitation, a vector-based system was developed that expressed short hairpin RNAs (shRNAs) that comprise a 19-29 bp stem with a loop size of 4-9 nucleotides. shRNAs are processed into siRNAs by an enzyme known as a “dicer” and exhibit specific gene silencing when expressed in human cells (See, for example, Brummelkamp, T. R., et al., Science, 296:550-553 (2002), Miyagishi, M., et al., Nat. Biotechnol., 20:497-500 (2002), Paddision, P. J., et al., Genes Dev., 16:948-958 (2002), Paul, C. P., et al., Nat. Biotechnol., 20:505-508 (2002) and Sui, G., et al., Proc. Natl. Acad. Sci. USA, 99:5515-5520 (2002)). Furthermore, it has been demonstrated that shRNA expression systems can be incorporated into chromosomes to establish stable cell lines or to create knockdown animals for studying gene function in vivo (See, Paddision, P. J., et al., Genes Dev., 16:948-958 (2002), Brummelkamp, T. R., et al., Cancer Cell, 2:243-247 (2002), Hemann, M. T., et al., Nat. Genet., 33:396-400 (2003), Tiscornia, G., et al., Proc. Natl. Acad. Sci., USA, 100:1844-1848 (2003), Barton, G. M., et al., Proc. Natl. Acad. Sci. USA, 99:14943-14945 (2002), Hasuwa, H., et al., FEBS Lett., 532:227-230 (2002), Kunath, T., Nat. Biotechnol., 21:559-561 (2003) and Rubinson, D. A, et al., Nat. Genet., 33:401-406 (2003)).

RNA polymerase III dependent promoter sequences are often chosen for expression of shRNAs. Unlike mRNAs produced by RNA pol II, transcripts produced by pol III do not have the 5′ cap and 3′ poly A tail, thereby allowing efficient processing of shRNA into siRNA by the dicer enzyme. Although the development of shRNA expression systems enables stable target knockdown in cells or animals, the constitutive activity of pol III dependent promoter sequences impose various restrictions on the use of shRNA expression systems. For example, constitutive expression of shRNAs that target genes with critical developmental functions result in embryonic lethality, which prevents the study of loss of function phenotypes in adult animals. In addition, the constitutive knockdown of a target with critical functions in cells or animals often trigger a compensatory response, which could alter the true consequence of gene silencing. Therefore, there is a need in the art for the controlled expression of shRNA that is useful for an unbiased analysis of the loss of function phenotype of essential genes in cells and animals.

Attempts have been made to develop tetracycline responsive pol III dependent promoter sequences. Two types of tetracycline-responsive derivatives of the human U6 shRNA promoter sequence are known in the art (See, Ohkawa, J., et al., Human Gene Therapy, 11:577-585 (2000)). In the tetracycline O1 (tetO1) type U6 promoter sequences, a type 1 tetracycline operator (tet operator) having the polynucleotide sequence of: actctatcattgatagagttat (SEQ ID NO: 1), was engineered between the proximal sequence element (PSE) and the TATA box. In the tetracycline O2 (tetO2) type U6 promoter, a type 2 tet operator having the polynucleotide sequence of ctccctatcagtgatagagaaa (SEQ ID NO: 5), was engineered between the TATA box and the transcriptional start site (TSS). Both the TATA box and PSE play essential roles in the transcription initiation by RNA polymerase III. It was reasoned that binding of the tetracycline repressor (tetR) to these modified U6 promoter sequences at positions adjacent to the TATA box or the PSE would interfere with small nuclear RNA (snRNA) activating protein complex (SNAPc) binding to the PSE and subsequently prevent transcription initiation. Both the tetO1 and tetO2 type promoter sequences have been shown to exhibit tetracycline-dependent transcriptional activity in a cell line that constitutively expresses tetR. However, the tetO1 appeared to have a better response to tetracycline treatment compared with the tetO2 type promoter in a transient transfection experiment (See, Ohkawa, J., et al., Human Gene Therapy, 11:577-585 (2000)). Controlled expression of shRNA using the tetO1 or the tetO2 type U6 promoters or using a human H1 shRNA promoter derivative with a design similar to that of the tetO2 type U6 promoter is also known in the art (See, Matsukura, S., et al., Nucleic Acid Res., 31:e77 (2003) and Czaudema, F., et al., Nucleic Acids Res., 31:e127 (2003)). Inducible knockdown of DNA methyltransferase (DNMT), beta catenin and PI3 kinase was achieved in stable cell lines using these systems. Although these pol III dependent promoter derivatives appeared to be tightly regulated in the literature, severe leakiness of these expression systems have been observed by the inventors of the present invention when the tetO1 promoter sequence was used to express a shRNA targeting a polynucleotide sequence of interest, such as luciferase. While not wishing to be bound by any theory, the inventors believe that it is likely that the binding of tetR to a single site on the U6 promoter is not sufficient to completely block the basal transcriptional activity of the promoter. When a potent shRNA is used, a slight leakiness of the system could lead to a significant reduction of the target protein. Tight regulation is one of the most critical and challenging requirements for all controlled expression systems. Depending on the target of interest, slight perturbation of the target level could be sufficient to cause phenotypic changes.

A third type of tetracycline responsive derivative of the human U6 shRNA promoter sequence is also known in the art. In this promoter sequence, both the tetO1 and tetO2 type promoters were engineered into the U6 promoter (See, Ohkawa, J., et al., Human Gene Therapy, 11:577-585 (2000)). The tetO1 operator was engineered between the PSE and the TATA box and the tetO2 operator engineered between the TATA box and TSS. However, Ohkawa et al. reported that the inclusion of both the tetO1 and tetO2 resulted in a complete loss of transcriptional activity for the U6 promoter sequence.

Thereupon, due to the potential limitations associated with the currently known inducible shRNA expression systems, there is a need in the art for a controlled shRNA expression system with minimal basal transcriptional activity. Specifically, there is a need for a tightly regulated promoter that can be used in such expression systems so as to improve the success rate in making inducible knockdown cell lines and non-human animals.

### SUMMARY OF THE INVENTION

In one embodiment, the present invention relates to a RNA pol III dependent promoter sequence. The promoter sequence can be a U6 promoter, a H1 promoter or a 7SK promoter. The promoter sequence of the present invention comprises a TATA element, a proximal sequence element (PSE) 5′ to the TATA element, a transcriptional start site (TSS) 3′ to the TATA element, a first tetracycline operator (first tet operator) located between the PSE and TATA element and a second tetracycline operator (second tet operator) located between the TATA element and TSS. In one aspect, the first tet operator is located between the TATA element and the PSE and does not form a portion of either the PSE or TATA element. In another aspect, the first tet operator is located between the TATA element and the PSE and forms of portion of one or both of the PSE or TATA element. The second tet operator is located between the TATA element and the TSS. In one aspect, the second tet operator is located between the TATA element and the TSS and does not form a portion of either the TSS or TATA element. In another aspect, the second tet operator is located between the TATA element and the TSS and forms of portion of one or both of the TSS or TATA element.

The polynucleotide sequence of the first tet operator and second tet operator can be identical or can be different. If the polynucleotide sequence of the first tet operator and the second tet operator are identical, the polynucleotide sequence can be selected from the group consisting of: actctatcattgatagagttat (SEQ ID NO: 1), tccctatcagtgatagaga (SEQ ID NO: 2), tccctatcagtgatagagacc (SEQ ID NO: 3), tccctatcagtgatagagagg (SEQ ID NO: 4) and ctccctatcagtgatagagaaa (SEQ ID NO: 5).

The polynucleotide sequence of the first tet operator and the second tet operator can be different from one another provided that when the first tet operator has the polynucleotide sequence of actctatcattgatagagttat (SEQ ID NO: 1), that the second tet operator does not have a polynucleotide sequence of ctccctatcagtgatagagaaa (SEQ ID NO: 5). The polynucleotide sequence of the first tet operator can be selected from the group consisting of: actctatcattgatagagttat (SEQ ID NO: 1), tccctatcagtgatagaga (SEQ ID NO: 2), tccctatcagtgatagagacc (SEQ ID NO: 3), tccctatcagtgatagagagg (SEQ ID NO: 4) and ctccctatcagtgatagagaaa (SEQ ID NO: 5). The polynucleotide sequence of the second tet operator can be selected independently from the group consisting of: actctatcattgatagagttat (SEQ ID NO: 1), tccctatcagtgatagaga (SEQ ID NO: 2), tccctatcagtgatagagacc (SEQ ID NO: 3), tccctatcagtgatagagagg (SEQ ID NO: 4) and ctccctatcagtgatagagaaa (SEQ ID NO: 5). Preferably, if the first tet operator has a polynucleotide sequence of tccctatcagtgatagagacc (SEQ ID NO: 2) the second tetracycline operator has the polynucleotide sequence of: actctatcattgatagagttat (SEQ ID NO: 1).

In another embodiment, the present invention relates to vectors comprising the herein described promoters. More specifically, the vectors of the present invention comprise at least one of the RNA pol III dependent promoter sequences described above that are operably linked to at least one polynucleotide sequence of interest. The at least one polynucleotide sequence of interest can be DNA or cDNA.

In another embodiment, the present invention relates to a eukaryotic cell that comprises at least one of the vectors described above.

In another embodiment, the present invention relates to transgenic non-human animals. Examples of transgenic non-human animals are mice, rats, dogs, cats, pigs, cows, goats, sheep, primates (other than humans) and guinea pigs. The transgenic non-human animals of the present invention comprise a transgene that comprises at least one polynucleotide sequence of interest that is operably linked to at least one of the RNA pol III dependent promoter sequences described herein. Transcription of the at least one polynucleotide sequence of interest produces an RNA molecule that modulates the expression of at least one target gene in said transgenic animal. The RNA molecule that is produced can be a small interfering RNA (siRNA) or a short hairpin RNA (shRNA).

In another embodiment, the present invention relates to methods of producing a transgenic non-human animal. In one aspect, a transgenic non-human animal can be produced pursuant to the following method. The first step of the method involves introducing a transgene into a fertilized oocyte of a non-human animal. This transgene comprises at least one polynucleotide sequence of interest that is operably linked to at least one of the RNA pol III dependent promoter sequences described herein. Transcription of the at least one polynucleotide sequence of interest produces an RNA molecule that modulates the expression of at least one target gene in said transgenic animal. The RNA molecule that is produced can be siRNA or shRNA. The next step in the method involves allowing the fertilized oocyte to develop into an embryo. The next step involves transferring the embryo into a pseudopregnant female non-human animal. The next step involves allowing the embryo to develop to term. The next step involves identifying the transgenic non-human animal containing the polynucleotide sequence of interest.

In another aspect, the transgenic non-human animal can be produced pursuant to the following method. The first step of the method involves introducing a transgene into an embryonic stem cell of a non-human animal. This transgene comprises at least one polynucleotide sequence of interest that is operably linked to at least one of the RNA pol III dependent promoter sequences described herein. Transcription of the at least one polynucleotide sequence of interest produces an RNA molecule that modulates the expression of at least one target gene in said transgenic animal. The RNA molecule that is produced can be a siRNA or shRNA. The next step in the method involves introducing said non-human embryonic stem cell into a blastocyst. The next step in the method involves implanting the resulting blastocyst into a pseudopregnant female non-human animal. The next step in the method involves allowing the non-human animal to give birth to a chimeric non-human animal. The next step involves breeding the chimeric non-human animal to produce a transgenic non-human animal containing said transgene.

In another embodiment, the present invention relates to a method for inducing transcription of at least one polynucleotide sequence of interest in an eukaryotic cell. In this method, when transcription is induced, the at least one polynucleotide sequence of interest produces at least one RNA molecule that modulates the expression of at least one target gene in the eukaryotic cell. The first step of the method involves providing an eukaryotic cell expressing the tetR protein. Once an eukaryotic cell has been provided, the next step is transforming or transfecting this cell with at least one vector, such as one of the vectors previously described herein. For example, the vector may contain at least one polynucleotide sequence of interest that is operably linked to at least one RNA pol III dependent promoter sequence described herein. The next step in the method involves contacting the cell with an inducing agent. The inducing agent binds to a tet repressor protein and causes the promoter sequence to transcribe the polynucleotide sequence of interest. Transcription of the polynucleotide sequence produces at least one RNA molecule that modulates the expression of at least one target gene in the cell. The inducing agent used in the above described method can be doxycycline, tetracycline or a tetracycline analogue. Additionally, the RNA molecule produced in the above described method can be siRNA or shRNA.

Optionally, the method described above can further comprise the step of transforming the eukaryotic cell with a second vector that contains a polynucleotide sequence operably linked to a promoter, wherein said polynucleotide sequence encodes a tet repressor that binds to at least one tet operator of the promoter.

Optionally, the at least one vector used in the above method can further contain a second polynucleotide sequence of interest. In one aspect, this second polynucleotide sequence can be operably linked to a second promoter sequence and can encode a tet repressor protein that binds to at least one of the tet operators of the promoter.

In a second aspect, this second polynucleotide sequence can be linked in tandem with the first polynucleotide sequence of interest. In this second aspect, when the cell is contacted with an inducing agent, the inducing agent binds to a tet repressor protein and the promoter causes the transcription of each of the first and second polynucleotide sequences of interest. Specifically, the transcription of the first polynucleotide sequence produces a first RNA molecule that modulates the expression of a first target gene and the transcription of the second polynucleotide sequence produces a second RNA molecule that modulates the expression of a second target gene.

In one embodiment of the invention, the at least one of the polynucleotide sequence of interest encodes a tyrosinase.

In a third aspect, the at least one vector not only contains a second polynucleotide sequence of interest that is linked in tandem with the first polynucleotide sequence of interest, but also a third polynucleotide sequence that is operably linked to a second promoter sequence. This third polynucleotide sequence encodes a tet repressor protein that binds to at least one of the tet operators of the promoter. In this third aspect, when the cell is contacted with an inducing agent, the inducing agent binds to a tet repressor protein and the promoter sequence causes the transcription of each of the first and second polynucleotide sequences of interest. Specifically, the transcription of the first polynucleotide sequence produces a first RNA molecule that modulates the expression of a first target gene and the transcription of the second polynucleotide sequence produces a second RNA molecule that modulates the expression of a second target gene.

### BRIEF DESCRIPTION OF THE FIGURES

FIG. 1 shows the sequence alignment of U6 promoter variants. U6 is the wildtype human U6 promoter (SEQ ID NO: 6). O1 (SEQ ID NO: 7) or O2 (SEQ ID NO: 8) is the O1 and O2 type human U6 promoter. O1O2_{—}1 (SEQ ID NO: 9), O1O2_{—}2 (SEQ ID NO: 10), O1O2_{—}3 (SEQ ID NO: 11), O1O2_{—}4 (SEQ ID NO: 12), O1O2_{—}5 (SEQ ID NO: 13), and O1O2_{—}6 (SEQ ID NO: 14) are U6 promoter variants with both O1 and O2 type tet operators. 2O2 (SEQ ID NO: 15) is the U6 promoter variant with two O2 type tet operators. The underscored italic sequence represents the O2 type tet operator. The underscored non-italic sequence represents the O1 type tet operator.

FIGS. 2A, 2B and 2C show the transcriptional activity and tetracycline response of U6 promoter variants.

FIGS. 3A, 3B and 3C show tetracycline dependent knockdown of an endogenous gene in stable cell lines using the 2O2 expression system.

FIGS. 4A, 4B and 4C show a comparison of the O1 and 2O2 expression system in making stable cell lines.

FIG. 5A shows the Hif1α protein levels in D54_Luc, D54_Hif25 and D54_Hif18 cells. Cells were incubated in the presence or absence of 1 μg/ml doxycycline. After thirty-six hours, cells were either untreated (N) or subjected to hypoxia treatment (H) for an additional sixteen hours. The cells were lysed and analyzed by western blotting using antibodies against Hif1α (upper panel) or Hif1β (lower panel). FIG. 5B shows the activity of the Hif1 reporter (1×HRE) or the constitutive reporter (pGL3) in D54_Luc, D54_Hif25 and D54_Hif18 cells. Cells were transfected with either pGL3-control/pRL-TK (left panel) or 1×HRE/pRL-TK plasmids (right panel) in the presence or absence of 1 μg/ml doxycycline. Thirty-six hours following transfection, cells were subjected to hypoxia treatment. Luciferase activities were determined sixteen hours after hypoxia treatment. FIG. 5C shows the mRNA levels of Hif1 target genes PGK1 and LDH in D54_Luc and D54_Hif25 cells. Cells were incubated in the presence or absence of 1 μg/ml doxycycline. After thirty-six hours, cells were either untreated (N) or subjected to hypoxia treatment (H) for an additional sixteen hours. Total RNA were prepared and used in quantitative PCR for analyzing the level of the indicated genes.

FIG. 6A shows the average Hif1α mRNA level and standard deviation (SD) in three D54_Hif25 (Hif) or D54_Luc (Luc) derived subcutaneous tumors treated with doxycycline. Mice bearing 200-300 mm³ tumors were supplied with doxycycline (1 mg/ml) for 3, 6, 9 or 12 days. Tumors were collected at the end of treatment and Hif1α mRNA levels were determined by QPCR. Tumors from mice without Dox treatment were used as controls. FIG. 6B shows the average Hif1α mRNA level in four D54-Hif25 or D54_Luc derived tumors excised from mice treated with water (Control) or doxycycline (Dox) for 45 days. Hif1α mRNA level was determined by QPCR. FIG. 6C shows the average Hif1α expression levels and standard deviation of the same tumor samples from B). The Hif1α expression level was examined by IHC, and quantified using AxioVision 4 (Zeiss).

FIG. 7A shows the average tumor size and standard error (SE) produced in subcutaneous tumors generated in 15 mice using D54-Hif25 (Hif25) or D54_Luc (Luc) cells and treated with and without doxycycline. After tumors reached the average size of 190 mm³, tumor-bearing mice were randomized and divided into two groups. Each group was supplied with drinking water containing doxycycline (Dox) or without doxycycline (control). Tumor sizes were measured twice/week using microcaliper. FIG. 7B shows the average tumor size and standard error (SE) of 8 mice in subcutaneous tumors generated using D54-Hif18 (Hif18) or D54_Luc (Luc) cells and treated with and without doxycycline. After tumors reached the average size of 150 mm³, tumor-bearing mice were randomized and divided into two groups. Each group was supplied with drinking water containing doxycycline (Dox) or without doxycycline (control). Tumor sizes were measured twice/week using microcaliper.

FIGS. 8A and 8B show mice born from embryos injected with the 2O2-Tyr731 transgene that exhibit different degrees of coat color change compared to the wild type mice. FIG. 8A shows a lighter coat color of one F0 of the founders compared to the darker coat color of the wild type mouse. FIG. 8B shows three pups that are white in color compared to the darker color of the F1 pups. The white colored pups are positive for the 2O2-Tyr731 transgene (SEQ ID NO: 48).

### DETAILED DESCRIPTION OF THE INVENTION

### Definitions and Other Terms

As used herein, the term “gene” refers to a polynucleotide sequence that undergoes transcription as a result of promoter activity. A gene may encode for a particular polypeptide, or alternatively, code for a RNA molecule. A gene can include one or more introns and/or exons and/or one or more regulatory and/or control sequences.

As used herein, the term “inducing agent” refers to an any compound that binds with specificity to a tet repressor protein, including, but not limited to, tetracycline, doxycycline or a tetracycline analogue.

As used herein, the terms “modulation” or “modulating” as used interchangeably herein, refer to both upregulation (i.e., activation or stimulation (e.g., by agonizing or potentiating)) and downregulation (i.e. inhibition or suppression (e.g., by antagonizing, decreasing or inhibiting)).

As used herein, the term “non-human animal” includes all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at a subcellular level, such as by targeted recombination or microinjection or infection with recombinant virus.

Mice are often used for transgenic animal models because they are easy to house, relatively inexpensive, and easy to breed. However, other non-human transgenic mammals may also be made in accordance with the present invention such as, but not limited to, primates, mice, goat, sheep, rabbits, dogs, cows, cats, guinea pigs and rats. Transgenic animals are those which carry a transgene, that is, a cloned gene introduced and stably incorporated which is passed on to successive generations.

As used herein, the term “operably linked” refers to a juxtaposition wherein the components so described are in a relationship permitting them to function in their intended manner. For example, a polynucleotide sequence of interest may be positioned adjacent another polynucleotide sequence that directs transcription or transcription and translation of the introduced polynucleotide sequence of interest (i.e., facilitates the production of, e.g., a polypeptide or a polynucleotide encoded by the introduced sequence of interest). A promoter is considered operably linked to a coding sequence if the promoter effects the transcription or expression of the coding sequence.

As used herein, the term “polynucleotide” means a polymeric form of nucleotides of any length, either ribonucleotides or deoxyribonucleotides. This term refers only to the primary structure of the molecule. Thus, the term includes double- and single-stranded DNA, as well as double- and single-stranded RNA. It also includes modifications, such as methylation or capping and unmodified forms of the polynucleotide. The terms “polynucleotide,” “oligomer,” “oligonucleotide,” and “oligo” are used interchangeably herein.

As used herein, the term “polynucleotide sequence of interest” refers to any DNA, cDNA, genomic DNA, nucleic acid analogs and synthetic DNA that is capable of expressing a RNA molecule, such as, but not limited to, small interfering RNA (siRNA) or short hairpin RNA (shRNA), or a protein or other molecule in a target cell (i.e., that is capable of the production of the protein or other biological molecule in a target cell). The DNA may be double-stranded or single-stranded, and if single stranded, may be the coding (sense) strand or non-coding (anti-sense) strand. The polynucleotide sequence of interest is generally operably linked to other polynucleotide sequences needed for expression, such as at least one promoter sequence. Any polynucleotide sequence of interest can be used in the present invention. Examples of polynucleotide sequences that can be used in the present invention include, but are not limited to, polynucleotide sequences to knock-out the mouse IRAK4 gene, such as, ggaagaaauuagcaguagc ucucuugaa gcuacugcuaauuucuuccuu (SEQ ID NO: 16), which can be used in shRNA methods, polynucleotide sequences to knock-out the human STK33 gene, such as, gggcauuucucagagaaugtt (SEQ ID NO: 17) and ttcccguaaagagucucuuac (SEQ ID NO: 18), each of which can be used in siRNA methods, polynucleotide sequences that encode a NFKB inhibitor Ras-like 1 (also known as “NKIRAS1”) protein or knock-out a NKIRAS1 gene (cDNA encoding a human NKIRAS1 protein can be found in GenBank as Accession No. NM-020345), polynucleotide sequences that encode a hypoxia-inducible factor 1, alpha subunit (a basic helix-loop-helix transcription factor and also known as “HIF1A”) protein or knock-out a HIF1A gene (cDNA encoding a human HIF1A protein can be found in GenBank as Accession No. NM_{—}001530), polynucleotide sequences that encode genomic chromosomes or knock-out a genomic chromosome, such as, but not limited to a chromosome 8 genomic contig (genomic DNA encoding a human chromosome 8 genomic contig can be found in GenBank as Accession No. NT_{—}023736.16), polynucleotide sequences that encode a member of the kinase family or that knock-out a gene that encodes a member of a kinase family (examples of members of a kinase family, include, activin A receptor type II-like proteins (also known as “ACVRL1”) (DNA encoding a human ACVRL1 protein can be found in GenBank as Accession No. NM_{—}000020) or ATM proteins (DNA encoding a human ATM protein can be found in GenBank as Accession No. NM_{—}000051)), polynucleotide sequences that encode tumor suppressor proteins or knock-out a gene that encodes a tumor suppressor protein (examples of tumor suppressor proteins include, the p53 protein (DNA encoding a human p53 protein can be found in GenBank as Accession No. NM_{—}000546) or a human retinoblastoma protein (DNA encoding a human retinoblastoma protein can be found in GenBank as Accession No. M15400)), polynucleotide sequences that encode transcriptional factors or that knock-out a gene that encodes a transcriptional factor (an example of a transcriptional factor, includes, the myc protein (DNA encoding a human myc protein can be found in GenBank as Accession No. M13228)), polynucleotide sequences that encode Sam11 GTPases or that knock-out a Sam11 GTPase gene (an example of Sam11 GTPases includes the Ras protein (DNA encoding a human Ras protein can be found in GenBank as Accession No. NM_{—}033360)), polynucleotide sequences that encode E3 ligases or that knock-out a gene encoding a E3 ligase (an example of a E3 ligase includes, the SKP2 protein (DNA encoding a human SKP2 protein can be found in GenBank as Accession No. NM_{—}032637)), etc.

As used herein, the term “polypeptide” and “protein” are used interchangeably herein and indicate at least one molecular chain of amino acids linked through covalent and/or non-covalent bonds. The terms do not refer to a specific length of the product. Thus peptides, oligopeptides and proteins are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide.

As used herein, the term “target gene” refers to a polynucleotide sequence, such as, but not limited to, a polynucleotide sequence of interest that encodes a polypeptide of interest or alternatively, a RNA molecule of interest, such as, but not limited to siRNA or shRNA. The target gene can be an “essential” gene required for continued cell viability whose function is to be shut-off by the methods of the present invention. The term “target gene” can also refer to a gene to be knocked-out according to the methods described herein.

As used herein, the term “tetracycline analogue” refers to any compound that is related to tetracycline or doxycycline and that binds with specificity to a tet repressor protein. The dissociation constant of such analogues should be at least 1×10⁻⁶ M, preferably greater than 1×10⁻⁹ M. Examples of tetracycline analogues are discussed in Hlavka et al., “The Tetracyclines,” in Handbook of Experimental Pharmacology 78, Blackwood et al. (eds), New York (1985) and Mitschef (“The Chemistry of Tetracycline Antibiotics,” Medicinal Res. 9, New York (1978), which is herein incorporated by reference.

As used herein, the terms “tetracycline repressor protein,”, “tet repressor protein”, and “tetR”, which are all used interchangeably herein, refer to a polypeptide that 1) exhibits specific binding to an inducing agent; 2) exhibits specific binding to at least one tet operator sequence when the tetracycline repressor protein is not bound by an inducing agent; and/or 3) is capable of being displaced or competed off from a tetracycline operator by an inducing agent. The term “tetracycline repressor protein” includes naturally-occurring (i.e., native) tetracycline repressor protein polypeptide sequences and functional derivatives thereof.

As used herein, the term “regulatory sequences” refer to those sequences normally associated with (for example, within 50 kb of) the coding region of a locus which affect the expression of a polynucleotide (including transcription of a gene, and translation, splicing, stability, or the like of a messenger RNA). Regulatory sequences include, for example, promoters, enhancers, splice sites and polyadenylation sites.

As used herein, the term “control sequence” refers to polynucleotide sequences which are necessary to effect the expression of coding sequences to which they are ligated. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is necessary for expression, and may also include additional components whose presence is advantageous, for example, leader sequences and fusion partner sequences.

As used herein, the term “transgene” refers to a polynucleotide sequence (encoding, e.g., one of the polypeptides, or an antisense transcript thereto) which has been introduced into a cell. A transgene could be partly or entirely heterologous, i.e., foreign, to the transgenic animal or cell into which it is introduced, or, homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected polynucleotide sequence.

As used herein, the term “vector” refers to a vehicle by which a polynucleotide or DNA sequence is introduced into the cell. It is not intended to be limited to any specific sequence. The vector could itself be the polynucleotide or DNA sequence that modulates the endogenous gene or could contain the polynucleotide sequence that modulates the endogenous gene. Thus, the vector could be simply a linear or circular polynucleotide containing essentially only those sequences necessary for modulation, or could be these sequences in a larger polynucleotide or other construct such as a DNA or RNA viral genome, a whole viron, or other biological construct used to introduce the critical nucleotide sequences into a cell. It is also understood that the phrase “vector construct”, “recombinant vector” or “construct” may be used interchangeably with the term “vector” herein.

As used herein, the singular forms “a,” “an” and “the” include plural-reference unless the context clearly dictates otherwise. Unless defined otherwise, all technical and scientific terms used herein have the same meaning as commonly understood to one those of skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to those of skill in the art to which this invention belongs. Although any methods, devices and materials similar or equivalent to those described herein can be used in the practice or testing of the invention, representative methods, devices and materials are now described.

Tetracycline Resistance Operons

Although not critical, information relating to tetracycline resistance (tet) operons in bacteria is briefly provided herein to help facilitate the understanding of the present invention.

In a tet operon, a polynucleotide sequence of interest and a gene encoding the tet repressor protein (tetR) are both under the control of the same operator elements. In the absence of an inducing agent, the tet repressor protein binds to the operator sequence, thereby sterically preventing the adjacent promoter sequence from interacting with transcription activators, such as RNA polymerase. Thus, transcription of the polynucleotide sequence of interest is blocked. When the level of the inducing agent within the bacterium increases, the agent binds to the tet repressor protein preventing it from binding to the operator sequence. As a result, the polymerase is able to bind to the promoter sequence and the polynucleotide sequence is transcribed.

Promoters of the Present Invention

In one embodiment, the present invention relates to RNA pol III dependent promoter sequences. Preferably, the RNA pol III dependent promoter sequences of the present invention are inducible, meaning that such promoters are inducible promoters. As used herein, the term “inducible” or “inducible promoter(s)”, both of which are used interchangeably herein, refers to the fact that the promoter sequences of the present invention are activated under a specific set of chemical conditions. These specific conditions are the presence of an inducing agent that binds to the tet repressor protein. For example, in the present invention, when an inducing agent is present, the promoter sequence of the present invention is activated and transcription of a polynucleotide sequence of interest, which is operably linked to said promoter sequence, occurs. The present invention contemplates that any RNA pol III dependent promoter sequence can be used herein, including, but not limited to the U6 promoter sequence, H1 promoter sequence or 7SK promoter sequence.

The promoter sequences of the present invention comprise a TATA element, a proximal sequence element (PSE) that is located 5′ to the TATA element, a transcriptional state site (TSS) that is located 3′ to the TATA element, at least one first tetracycline operator (first tet operator) and at least one second tetracycline operator (second tet operator). The promoter sequences of the present invention contain at least two tetracycline operators but promoter sequences containing more than two tetracycline operators are also contemplated as being within the scope of the present invention.

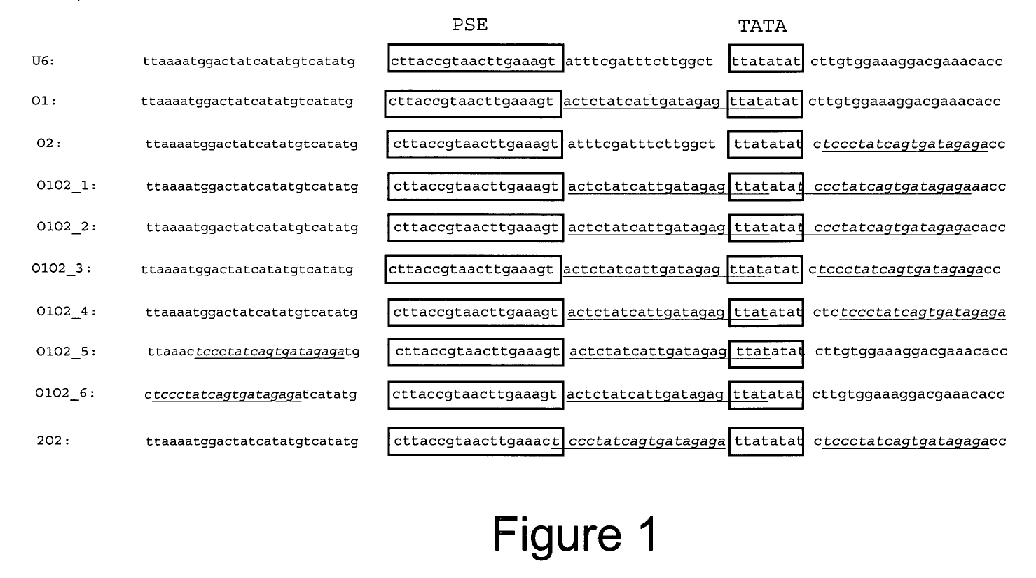

In the promoter sequences of the present invention, the first tet operator is located between the TATA element and the PSE (See FIG. 1). In one aspect, the first tet operator is located between the TATA element and the PSE and does not form a portion of either the PSE or TATA element. In another aspect, the first tet operator is located between the TATA element and the PSE and forms a portion of one or both of the PSE or TATA element. The second tet operator is located between the TATA element and the TSS (See FIG. 1). In one aspect, the second tet operator is located between the TATA element and the TSS and does not form a portion of either the TSS or TATA element. In another aspect, the second tet operator is located between the TATA element and the TSS and forms a portion of one or both of the TSS or TATA element. The arrangement of these elements must not substantially interfere with the ability of the promoter sequence to direct the transcription of a downstream polynucleotide sequence of interest or the translation of the gene product, if so desired. Moreover, procedures for synthesizing or purifying promoter sequences, operators and other polynucleotide sequences are well known to those of skill in the art and can be employed for constructing vectors (which will be described in more detail herein) with appropriately arranged elements as described in Sambrook et al., Molecular Cloning: A Laboratory Manual, 2^{nd} ed., Cold Spring Harbor Press (1989).

By engineering the at least two tetracycline operators within the specific locations of the promoter sequences described herein, the inventors of the present invention have found that when the promoter sequences of the present invention are operably linked to at least one polynucleotide sequence of interest, that the promoter sequences exhibit lower basal transcriptional activity compared to other inducible pol III dependent promoters known in the art. Consequently, as a result of the promoters of the present invention exhibiting tighter regulation, these promoter sequences greatly improve the success rate in making inducible knockdown cell lines.

The polynucleotide sequences of the first tet operator and second tet operator can be the same (i.e., identical) or can be different. The first tet operator and second tet operator can have any polynucleotide sequence provided that said polynucleotide sequence is such that it allows for the binding of a tet repressor protein to one and/or both of said operators in the absence of an inducing agent. For example, if the polynucleotide sequence of the first tet operator and the second tet operator are identical, the polynucleotide sequences of said operators can be selected from the group consisting of: actctatcattgatagagttat (SEQ ID NO: 1), tccctatcagtgatagaga (SEQ ID NO: 2), tccctatcagtgatagagacc (SEQ ID NO: 3), tccctatcagtgatagagagg (SEQ ID NO: 4) and ctccctatcagtgatagagaaa (SEQ ID NO: 5).

As mentioned previously, the polynucleotide sequence of the first tet operator and second tet operator do not have to be identical and can be different from one another. Again, as mentioned previously, the first tet operator and second tet operator can have any polynucleotide sequence provided that said polynucleotide sequence is such that it allows for the binding of a tet repressor protein to one and/or both of said operators in the absence of an inducing agent. For example, the polynucleotide sequence of the first tet operator can be selected from the group consisting of: actctatcattgatagagttat (SEQ ID NO: 1), tccctatcagtgatagaga (SEQ ID NO: 2), tccctatcagtgatagagacc (SEQ ID NO: 3) tccctatcagtgatagagagg (SEQ ID NO: 4) and ctccctatcagtgatagagaaa (SEQ ID NO: 5). The polynucleotide sequence of the second tet operator can be selected independently from the group consisting of: actctatcattgatagagttat (SEQ ID NO: 1), tccctatcagtgatagaga (SEQ ID NO: 2), tccctatcagtgatagagacc (SEQ ID NO: 3) tccctatcagtgatagagagg (SEQ ID NO: 4) and ctccctatcagtgatagagaaa (SEQ ID NO: 5). However, if the first tet operator has a polynucleotide sequence of actctatcattgatagagttat (SEQ ID NO: 1), then the second tet operator must not have a polynucleotide sequence of ctccctatcagtgatagagaaa (SEQ ID NO: 5). Nonetheless, it is preferred that the first tet operator have a polynucleotide sequence of tccctatcagtgatagagacc (SEQ ID NO: 2) and that the second tetracycline operator has the polynucleotide sequence of: actctatcattgatagagttat (SEQ ID NO: 1).

Vectors of the Present Invention

The promoter sequences of the present invention will typically be incorporated into at least one expression vector (such as, but not limited to, a plasmid, virus or phage). Large numbers of suitable vectors are known to those of skill in the art and are commercially available and can be used in the present invention. The following vectors are provided by way of example. Bacterial: pINCY (Incyte Pharmaceuticals Inc., Palo Alto, Calif.), pSPORT1 (Life Technologies, Gaithersburg, Md.), pQE70, pQE60, pQE-9 (Qiagen) pBs, phagescript, psiX174, pBluescript SK, pBsKS, pNH8a, pNH16a, pNH18a, pNH46a (Stratagene); pTrc99A, pKK223-3, pKK233-3, pDR540, pRIT5 (Pharmacia); Eukaryotic: pWLneo, pSV2cat, pOG44, pXT1, pSG (Stratagene) pSVK3, pBPV, pMSG, pSVL (Pharmacia). However, any other vector may be used as long as it is replicable and viable in a host. If desired, large amounts of vector DNA can be generated (for example, by transferring the vector into bacteria that make the repressor protein).

The expression vector will also contain at least one polynucleotide sequence of interest. This polynucleotide sequence of interest can be derived from any source and may be inserted into the vector by a variety of procedures that are known to those of skill in the art. Generally, the polynucleotide sequence of interest can be inserted into appropriate restriction endonuclease sites. Such procedures and others are deemed to be within the scope of those of skill in the art. The expression vector can also contain an origin of replication, a ribosome binding site for translation initiation and a transcription terminator. The vector may also include appropriate sequences for amplifying expression. In addition, the vector can contain one or more selectable marker sequences, such as antibiotic resistance genes (e.g., ampicillin, hygromycin, G418), β-galactosidase, or other gene products that can be used for the selection of cells containing the vector.

As mentioned briefly above, the vector can contain at least one polynucleotide sequence of interest. The vector can contain two or more polynucleotide sequences of interest wherein each polynucleotide sequence is operably linked to its own promoter sequence. The promoter sequence for each polynucleotide sequence can be the same or different provided that at least one polynucleotide sequence is operably linked to at least one promoter sequence of the present invention. For example, the vector may contain a first promoter sequence operably linked to a first polynucleotide sequence of interest and a second promoter sequence operably linked to a second polynucleotide sequence. The first and second promoter sequences can each be the promoter sequences of the present invention or can be different promoter sequences provided that at least one of the first or second promoter sequences is the promoter sequence of the present invention. Examples of suitable promoter sequences that are not the promoter sequences of the present invention and can be operably linked to either the first or second polynucleotide sequences of interest include, but are not limited to, LTR or the SV40 promoter, the E. coli lac or trp, the phage lambda P sub L promoter and other promoters known to those of skill in the art. Other regulatory and/or control sequences can be included with said promoter as well. Alternatively, the first and second polynucleotide sequences of interest can be linked in tandem and operably linked in an appropriate fashion to the promoter sequence of the present invention.

The vectors described herein can be introduced (i.e. transformed or transfected) into host cells, such as mammalian (such as, but not limited to, simian, canine, feline, bovine, equine, rodent, murine, etc.) or non-mammalian (such as, but not limited to, insect, reptile, fish, avian, etc.) cells, using any method known to those of skill in the art including, but not limited to, electroporation, calcium phosphate precipitation, DEAE dextran, lipofection, and receptor mediated endocytosis, polybrene, particle bombardment, and microinjection. Alternatively, the vector can be delivered to the cell as a viral particle (either replication competent or deficient). Examples of viruses useful for the delivery of nucleic acid include, but are not limited to, lentivirus, adenoviruses, adeno-associated viruses, retroviruses, Herpesviruseses, and vaccinia viruses. Other viruses suitable for delivery of polynucleotide sequences into cells that are known to those of skill in the art may be equivalently used in the present invention.

The engineered host cells can be cultured in conventional nutrient media modified as appropriate for activating the promoter sequences, selecting transfected cells, etc. The culture conditions, such as temperature, pH and the like, are those previously used with the host cell selected for expression, and will be apparent to those of skill in the art.

Preferably, the recombinant vector is transferred, transformed or transfected into a host cell that has been engineered to express the tet repressor protein. There are a number of ways to engineer host cells to express the tet repressor protein. For example, one way is to operably link the tet repressor gene sequence to a promoter sequence and then to incorporate this into the vector containing the promoter sequence of the present invention operably linked to the polynucleotide sequence of interest in tandem and then transfer, transform or transfect the vector into the host cells. In such an expression vector, the tet repressor sequence will be operably linked to a second promoter sequence (the first promoter sequence being the promoter sequence containing the at least two tet operators and operably linked to the polynucleotide sequence of interest). If the recombinant vector contains at least two polynucleotide sequences of interest, then the tet repressor sequence will be operably linked to a “second” or “third” promoter sequence depending upon whether the polynucleotide sequences of interest are each operably linked to a single promoter or operably linked to separate promoters. Alternatively, cells may be transformed or transfected with a separate recombinant vector containing the tet repressor sequence operable linked to a promoter sequence prior to the transfer of the vector containing the promoter sequence of the present invention operably linked to the polynucleotide sequence of interest. Examples of suitable “a promoter sequence” or “second” or “third” promoter sequences that can be operably linked to the tet repressor sequence include, but are not limited to, LTR or the SV40 promoter, the E. coli lac or trp, the phage lambda P sub L promoter and other promoters known to control expression of tet repressor sequences. Other regulatory and/or control sequences can be included with said promoter as well.

The engineered host cells containing the incorporated vector(s) can be identified using hybridization techniques that are well known to those of skill in the art or by using the polymerase chain reaction (PCR) to amplify specific polynucleotide sequences. If the polynucleotide sequence transferred to the cells produces a protein that can be detected, for example, by means of an immunological or enzymatic assay, then the presence of recombinant protein can be confirmed by introducing tetracycline into cells and then performing the assays either on the medium surrounding the cells or on cellular lysates.

As discussed previously herein, in the absence of any inducing agent, host cells transformed or transfected with the recombinant vectors containing the promoter sequences described herein exhibit lower basal transcriptional activity compared to other inducible pol III dependent promoter sequences known in the art. Nonetheless, transcription of the at least one polynucleotide sequence of interest incorporated into the host cells can be achieved by using an inducing agent. The amount of inducing agent to be added to the host cells to achieve the transcription of the at least one polynucleotide sequence of interest can be readily determined by those of skill in the art. Once induced, transcription of at least one polynucleotide sequence produces a RNA molecule. Preferably, this RNA molecule modulates the expression of a target gene in the host cell. If the host cell has been transformed or transfected with a recombinant vector containing more than one polynucleotide sequence of interest, each polynucleotide sequence will produce a RNA molecule. Preferably, these RNA molecules will modulate the expression of more than one target gene in a host cell. For example, if said host cells are transformed or transfected with two polynucleotide sequences of interest, the first polynucleotide sequence of interest can, as a result of transcription, produce a first RNA molecule that modulates the expression of a first target gene in said cell. The second polynucleotide sequence of interest can also, as a result of transcription, produce a second RNA molecule that modulates a second target gene in said cell. Preferably, said second target gene is different than the first target gene. Also, preferably, the modulation accomplished by the first and/or second RNA molecule is an inhibition or suppression of the first and/or second target gene. However, the present invention does contemplate that one RNA molecule might inhibit or suppress a first target gene while the second RNA molecule might activate or stimulate a second target gene.

Small Interfering RNA and Short Hairpin RNA

A brief description of siRNA and shRNA is provided to help facilitate the understanding of the present invention. Several U.S. and P.C.T. Patent Application Publications teach preferred methods for designing, synthesizing, purifying, and delivering siRNAs and shRNAs into cells. In particular, U.S. Patent Application Publication U.S. 2003/0148519, which is incorporated by reference herein in its entirety, provides compositions and methods for intracellular expression and delivery of siRNAs and shRNAs in mammalian cells; and U.S. Patent Application Publication U.S. 2002/0132788, which is incorporated by reference herein in its entirety, provides a process for delivering siRNAs into cells in vivo for the purpose of inhibiting gene expression in those cells.

Small interfering RNAs (siRNAs) are short intermolecular duplexes, generally composed of two distinct (sense and antisense) strands of RNA, each of approximately 21 nucleotides, that form approximately 19 basepairs, with single stranded 3′ overhands of 1-3, preferably 2 nucleotides. The base-paired regions of siRNAs generally substantially correspond, but are preferably exact to a “target gene” and its complement, in the RNA transcript to be targeted for degradation or translational inhibition.

The specific and necessary features of siRNAs required for inducing the efficient degradation or silencing of corresponding RNA transcripts have been investigated along with the features of the target gene within the targeted transcript. Methods for the design of effective siRNA's are described in Tuschl et al., Genes & Dev., 13:3191-3197 (1999) and Elbashir et al., EMBO J., 20:6877-6888 (2001), each of which are herein incorporated by reference.

For purposes of the present invention, the individual single-stranded RNAs comprising siRNAs are synthesized endogenously (within cells). The two complementary single strands must then anneal to form an RNA duplex—the siRNA. The annealing step also occurs endogenously. Endogenously synthesized single-stranded RNAs are synthesized by cellular RNA polymerases using the vectors described herein that contain the promoters of the present invention.

Short hairpin RNAs (shRNAs), are single-stranded RNAs with regions of self-complementarity that can pair with one another, allowing the single strand to fold into an intramolecular duplex with a stem-loop type structure. Although the unpaired loop region can theoretically be any size, it is advantageous for the loop to be small enough to readily allow the self-complementary sequences within the same single-stranded RNA to find each other and basepair. Preferred loop sizes are from 4 to 9 nucleotides, and larger, with loops of 5-8 nucleotides being most preferred. Generally the sequence of the loop is not important, however, it should not contain a palindromic sequence. Within the cell the loop of an shRNAs is cleaved and an intermolecular duplex, not unlike an siRNA, is formed. The stem region of the shRNA should generally contain approximately 19-29 base pairs, and generally 3′ end of the shRNA extending beyond the paired region is composed of multiple thymidylate residues. The base-paired regions of shRNAs generally correspond substantially, preferably exactly, to a target gene and its complement in the RNA transcript to be targeted for degradation, just as the base-paired region in siRNAs does.

Like the single strands of siRNAs, shRNAs can be can be synthesized either endogenously, or exogenously. Endogenously synthesized shRNAs are generally synthesized by cellular RNA polymerases using the vectors described herein that contain the promoters of the present invention.

Methods for Modulating Gene Expression in Non-Human Mammals

In another embodiment, the present invention relates to methods of modulating the expression of at least one target gene in at least one eukaryotic cell in a non-human animal. These methods involve inducing the transcription of a polynucleotide sequence of interest using the promoter sequences and recombinant vectors described herein. As discussed previously herein, the transcription of said polynucleotide sequence of interest produces at least one RNA molecule. Examples of RNA molecules that can be produced include, but are not limited to, siRNA or shRNA. These RNA molecules are then used to modulate the expression of at least one target gene in such cells.

The promoter sequences and vectors of the present invention described herein can be used in a variety of methods for modulating the expression of at least one target gene in a eukaryotic cell. More specifically, the method involves providing at least one eukaryotic cell and then transforming or transfecting said eukaryotic cell with at least one of the recombinant vectors described herein. The at least one polynucleotide sequence of interest contained within the recombinant vectors described herein, upon transcription preferably produces at least one RNA molecule that modulates the expression of at least one target gene in said cell. Depending upon the purpose intended, the at least one RNA molecule can either 1) activate or stimulate the target gene or 2) inhibit or suppress the target gene. For example, if a target gene in a eukaryotic cell is to be “knocked out”, then the RNA molecule produced may be siRNA or shRNA. It is known to those of skill in the art that siRNA or shRNA can be used to “knock-out” target genes. Therefore, the result of this modulation would be to inhibit or suppress the target gene. Methods for making polynucleotide sequences of interest that encode siRNA or shRNA are described herein.

Transgenic Non-Human Animals

In another embodiment, the present invention relates to transgenic non-human animals that contain the promoter sequences and vectors described herein as well as methods of making said animals. A variety of methods can be used to create the transgenic non-human animals of the present invention. For example, the generation of a specific alteration of a polynucleotide sequence of a target gene is one approach that can be used. Alterations can be accomplished by a variety of enzymatic and chemical methods used in vitro. One of the most common methods uses a specific oligonucleotide as a mutagen to generate precisely designed deletions, insertions and point mutations in a target gene. Secondly, a wildtype human gene and/or humanized non-human animal gene could be inserted by homologous recombination. It is also possible to insert an altered or mutant (single or multiple) human gene as genomic or minigene constructs using the promoter of the present invention.

Additionally, transgenic non-human animals can also be made wherein at least one endogenous target gene is “knocked-out”. The creation of knockdown animals allows those of skill in the art to assess in vivo function of the gene that has been “knocked-out”. The knock-out of at least one target gene may be accomplished in a variety of ways. One strategy that can be used to “knock-out” a target gene is by the insertion of artificially modified fragments of the endogenous gene by homologous recombination. In this technique, mutant alleles are introduced by homologous recombination into embryonic stem (ES) cells. The embryonic stem cells containing a knock out mutation in one allele of the gene being studied are introduced into a blastocyst. The resultant animals are chimeras containing tissues derived from both the transplanted ES cells and host cells. The chimeric animals are mated to assess whether the mutation is incorporated into the germ line. Those chimeric animals each heterozygous for the knock-out mutation are mated to produce homozygous knock-out mice. A second strategy that can be used to “knock-out” at least one gene involves using siRNA and shRNA and oocyte microinjection or transfection or microinjection into embryonic stem cells as described further herein. As mentioned previously herein, because the promoter sequences of the present invention exhibit tighter regulation, these promoter sequences greatly improve the success rate in making inducible knockdown cell lines and animals when compared to other promoter sequences known in the art.

To create a transgenic non-human animal having an altered version of a human target gene, a polynucleotide sequence of interest can be inserted into a non-human animal germ line using standard techniques of oocyte microinjection or transfection or microinjection into embryonic stem cells. Alternatively, if it is desired to knock-out or replace a endogenous gene, homologous recombination using embryonic stem cells or siRNA or shRNA using oocyte microinjection or transfection or microinjection of embryonic stem cells can be used as described herein.

For oocyte injection, at least one polynucleotide sequence of interest that is operably linked to the promoter of the present invention can be inserted into the pronucleus of a just-fertilized non-human animal oocyte. This oocyte is then reimplanted into a pseudopregnant foster mother. The liveborn non-human animal can then be screened for integrants by analyzing the animal's DNA (using polymerase chain reaction (PCR) for example) such as from the tail, for the presence of the polynucleotide sequence of interest. Chimeric non-human animals are then identified. The transgene can be a complete genomic sequence injected as a YAC or chromosome fragment, a cDNA, or a minigene containing the entire coding region and other elements found to be necessary for optimum expression.

Retroviral or lentiviral infection (See, Lois C, et al., Science, 295:868-872 (2002) (which teaches methods for transgenics using lentiviral transgenesis)) of early embryos can also be done to insert an altered gene. In this method, the altered gene is inserted into a retroviral vector which is used to directly infect mouse embryos during the early stages of development to generate a chimera, some of which will lead to germline transmission (Jaenisch, R., Proc. Natl. Acad. Sci. USA, 73: 1260-1264 (1976)).

Homologous recombination using embryonic stem cells allows for the screening of gene transfer cells to identify the rare homologous recombination events. Once identified, these can be used to generate chimeras by injection of at least one non-human animal blastocyst and a proportion of the resulting animals will show germline transmission from the recombinant line. This gene targeting methodology is especially useful if inactivation of the gene is desired. For example, inactivation of the gene can be done by designing a polynucleotide fragment which contains sequences from an exon flanking a selectable marker. Homologous recombination leads to the insertion of the marker sequences in the middle of an exon, inactivating the gene. DNA analysis of individual clones can then be used to recognize the homologous recombination events.

Alternatively, “knock-out” of a target gene can be accomplished using siRNA or shRNA. In one strategy, oocyte microinjection can be used as described herein. Specifically, a transgene comprising at least one polynucleotide sequence of interest that expresses at least one RNA molecule that is siRNA or shRNA and that is operably linked to at least one RNA pol III dependent promoter sequence of the present invention is prepared using the methods described herein. This transgene is introduced into a non-human animal fertilized oocyte, preferably, by injection. The fertilized oocyte is then allowed to develop into an embryo. The resulting embryo is then transferred into a pseudopregnant female non-human animal and then allowed to give birth. Liveborn non-human animals are then screened for chimeric animals that contain the transgene by obtaining a sample and analyzing the animal's DNA (using techniques such as PCR) and such chimeric non-human animals are identified. When these non-human animals are treated with an inducing agent, transcription is induced, the siRNA or shRNA expressed, and the target gene is repressed or “knocked-out”. In the absence of the inducing agent, the gene is not repressed or “knocked-out”.

In a second strategy, microinjection of embryonic stem cells can be used as described herein. Specifically, a transgene comprising at least one polynucleotide sequence of interest that expresses at least one RNA molecule that is siRNA or shRNA is operably linked to at least one RNA pol III dependent promoter sequence of the present invention is prepared using the methods described herein. This transgene is introduced into non-human animal embryonic stem cells which can be used to generate chimeras by introducing these embryonic stem cells, preferably by injection, into at least one non-human animal blastocyst. The resulting blastocyst is then implanted into a pseudopregnant female non-human animal and then allowed to give birth to a chimeric non-human animal. PCR can be used to identify the animals of interest. Liveborn non-human animals are then screened for chimeric animals that contain the transgene by obtaining and analyzing a sample of said animal's DNA (using techniques such as PCR) and such chimeric non-human animals are identified. This chimeric non-human animal can then be used in breeding to produce a transgenic non-human animal that stably contain this transgene within their genome. As with the previous method, when these non-human animals are treated with an inducing agent, transcription is induced, the siRNA or shRNA expressed, and the target gene is repressed or “knocked-out”. In the absence of the inducing agent, the gene is not repressed or “knocked-out”.

Methods of making transgenic animals are described, e.g., in Wall et al., J. Cell Biochem., June: 49(2), 113-20 (1992); Hogan, et al., in “Manipulating the mouse embryo”, A Laboratory Manual. Cold Spring Harbor Laboratory Press, Cold Spring Harbor, N.Y. (1992); in WO 91/08216 or U.S. Pat. No. 4,736,866 the disclosures of which are hereby incorporated by reference in their entirety.

By way of example, and not of limitation, examples of the present invention shall now be given.

### EXAMPLE 1

### Development of a Tightly Regulated U6 Promoter for shRNA Expression

a. Luciferase Assay

Luciferase reporter constructs, pGL-3 (Promega, Madison Wis.) and pRL-TK (Promega, Wisconsin) were transfected into cells using Lipofectamine 2000 (Invitrogen, Carlsbad, Calif.). Luciferase activity was determined using the Dual-Luciferase Assay System (Promega, Madison, Wis.).

b. Western Analysis

Cells were directly lysed on 6-well plates in 1× Laemmli sample buffer. Proteins were separated by SDS-PAGE, transferred to PVDF membrane, and western blotting was performed using antibodies against Chk1 (1:200, Santa Crutz Biotechnology, Santa Crutz, Calif. 95060), HIF-1 alpha (1:500, BD Bioscience, Palo Alto, Calif. 94303) or tetR (1:2000, Mo Bi Tec, Germany).

c. Cell Culture

D54-MG (a proprietary cell line owned by Abbott Laboratories) cells were grown in Dulbecco's modified Eagle's medium (DMEM) supplemented with 10% fetal bovine serum (FBS). HeLa-TREx cells (Invitrogen) were grown in minimum essential medium (MEM) supplemented with 10% FBS. H1299 (a proprietary cell line owned by Abbott Laboratories) cells were grown in RPMI 1640 medium supplemented with 10% FBS. All cells were maintained at 37° C. in an environment of 5% CO₂. The D54-MG-tetR cell lines were established by transfecting the D54-MG parental cell line with pcDNA6/TR (Invitrogen Corp., Carlsbad, Calif. 92008) and selected using 10 μg/ml of blasticidin.

d. Molecular Cloning

The human U6 promoter was synthesized using polymerase chain reaction (PCR). All PCR reactions were performed pursuant to the Advantage2 PCR Kit (BD Bioscience Clontech, Palo Alto, Calif.) using the following primers:

(SEQ ID NO: 19)
U6_1: gatcgaattccaggcaaaacgcaccacgtgacggagcgtgaccgcgcgccgagcgcgcgccaaggtcgggcagga.
(SEQ ID NO: 20)
U6_2: aacagccttgtatcgtatatgcaaatatgatggaatcatgggaaataggccctcttcctgcccgaccttggcgcg.
(SEQ ID NO: 21)
U6_3: atatacgatacaaggctgttagagagataattagaattaatttgactgtaaacacaaagatattagtataaaata.
(SEQ ID NO: 22)
U6_4: aaacataattttaaaactgcaaactacccaagaaattattactttctacgtcacgtattttatactaatatcttt.
(SEQ ID NO: 23)
U6_5: gcagttttaaaattatgttttaaaatggactatcatatgcttaccgtaacttgaaagtatttcgatttcttggct.
(SEQ ID NO: 24)
U6_6: tctagaagcttggtgtttcgtcctttccacaagatatataaagccaagaaatcgaaatact.

After being assembled using primers U6_{—}1, U6_{—}2, U6_{—}3, U6_{—}4, U6_{—}5 and U6_{—}6, the full length U6 promoter was amplified using the primer pair U6_{—}5′PCR (gatcgaattccaggcaaaacgcaccacgtg) (SEQ ID NO: 25) and U6_{—}3′PCR (tctagaagcttggtgtttcgtcctttccac) (SEQ ID NO: 26). The amplified PCR fragment was cloned into the EcoRI and HindIII sites of pBluescriptII (SK+) to create pU6.

Tetracycline regulated U6 promoter variants pU6_O1, pU6_{—O}2, pU6_{—O}1O2_{—}1, pU6_{—O}1O2_{—}2, pU6_{—O}1O2_{—}3, pU6_{—O}1O2_{—}4, pU6_{—O}1O2_{—}5, pU6_{—O}1O2_{—}6 and pU6_{—}2O2 were all generated by PCR modification of the U6 promoter. U6_{—}5′PCR was used as 5′ primer and the following primers were used as 3′ primers respectively:

(SEQ ID NO: 27)

| | |
|---|---|
| O1rev: | ggtgtttcgtcctttccacaagatatataactctatcaatg |
| | atagagtactttcaagttacggtaagcatatgata. |

(SEQ ID NO: 28)

| | |
|---|---|
| O2rev: | tttctctatcactgatagggagatatataaagccaagaaat |
| | cgaaatac. |

(SEQ ID NO: 29)

| | |
|---|---|
| O1O2_rev: | tctagaagcttggtgtttcgtcctttccacaagatatataa |
| | ctctatcaatgataga. |

(SEQ ID NO: 30)

| | |
|---|---|
| 0101_1: | ggtttctctatcactgatagggatatataactctatcaatg |
| | ata. |

(SEQ ID NO: 31)

| | |
|---|---|
| O1O2_2: | ggtgtctctatcactgatagggatatataactctatcaatg |
| | atagagtactttcaa. |

(SEQ ID NO: 32)

| | |
|---|---|
| O1O2_3: | ggtctctatcactgatagggagatatataactctatcaatg |
| | ataga. |

(SEQ ID NO: 33)

| | |
|---|---|
| O1O2_4: | tctctatcactgatagggagagatatataactctatcaatg |
| | atagagt. |

(SEQ ID NO: 34)

| | |
|---|---|
| O1O2_5: | ataactctatcaatgatagagtactttcaagttacggtaag |
| | catctctatcactgatagggaacataattttaaaactgcaa |
| | act. |

(SEQ ID NO: 35)

| | |
|---|---|
| O1O2_6: | ataactctatcaatgatagagtactttcaagttacggtaag |
| | catatgatctctatcactgatagggaattttaaaactgcaa |
| | acta. |

(SEQ ID NO: 36)

| | |
|---|---|
| 2O2: | ggtctctatcactgatagggagatatataatctctatcact |
| | gatagggagtttcaagttacggtaagcatatgatagtcc. |

Briefly, pU6_{—O}1 and pU6_{—O}2 were generated by PCR using pU6 as template and U6_{—}5′PCR and O1rev or pU6_{—O}2 as primers respectively. Tetracycline regulated U6 promoter variants pU6_{—O}1O2_{—}1, pU6_{—O}1O2_{—}2, pU6_{—O}1O2_{—}3, and pU6_{—O}1O2_{—}4, were all created by PCR using pU6_O1 as template, U6_{—}5′PCR as 5′ primer, and O1O2_{—}1, O1O2_{—}2, O1O2_{—}3, or O1O2_{—}4 as 3′ primers respectively. pU6_{—O}1O2_{—}5 and pU6_{—O}1O2_{—}6 were generated by two PCR steps. In the first step, pU6_{—O}1 was used as a template, the primer pairs U6_{—}5′PCR and O1O2_{—}5 or U6_{—}5′PCR and O1O2_{—}6 were used as primers respectively. In the second step, the PCR products from the first step were each used as a template, and U6_{—}5′PCR and O1O2_rev were used as primers. The U6 promoter variant with two O2 type tet operators, pU6_{—}2O2, was generated by PCR using pU6 as template and U6_{—}5′PCR and 2O2 as primers.

U6 promoter variants that express shRNAs targeting luciferase, or HIF-1α were designated as U6_luc, O1_luc, O2_luc, O1O2_luc1, O1O2_luc2, O1O2_luc3, O1O2_luc4, O1O2_luc5, O1O2_luc6, 2O2_luc, and 2O2_Hif1. These constructs were generated by PCR from each promoter variants using primers U6_{—}5′PCR and the following 3′ primers respectively with the exception that the primer O1O2_luc_rev was used to create both O1O2_luc5 and O1O2_luc6. The PCR fragments were then cloned into the EcoR I and Hind III site of pBluescript II (SK+).

(SEQ ID NO: 37)

| | |
|---|---|
| O1_luc: | gatcaaagcttaaaaaaggacatcacttacgctgagt |
| | ctcttgaactcagcgtaagtgatgtccggtgtttcgt |
| | cctttccacaa. |

(SEQ ID NO: 38)

| | |
|---|---|
| 02_luc: | tagaagcttaaaaaggacatcacttacgctgagtctc |
| | ttgaactcagcgtaagtgatgtcctttctctatcact |
| | gatag. |

(SEQ ID NO: 39)

| | |
|---|---|
| O1O2_luc_rev: | gatcaaagcttaaaaaaggacatcacttacgctgagt |
| | ctcttgaactcagcgtaagtgatgtccggtgtttcgt |
| | cctttccacaa. |

(SEQ ID NO: 40)

| | |
|---|---|
| O1O2_luc1: | gatcaaagcttaaaaaaggacatcacttacgctgagt |
| | ctcttgaactcagcgtaagtgatgtccggtttctcta |
| | tcactgataggg. |

(SEQ ID NO: 41)

| | |
|---|---|
| O1O2_luc2: | gatcaaagcttaaaaaaggacatcacttacgctgagt |
| | ctcttgaactcagcgtaagtgatgtccggtgtctcta |
| | tcactgataggg. |

(SEQ ID NO: 42)

| | |
|---|---|
| O1O2_luc3: | gatcaaagcttaaaaaaggacatcacttacgctgagt |
| | ctcttgaactcagcgtaagtgatgtccggtctctatc |
| | actgatagggag. |

(SEQ ID NO: 43)

| | |
|---|---|
| O1O2_luc4: | gatcaaagcttaaaaaaggacatcacttacgctgagt |
| | ctcttgaactcagcgtaagtgatgtcctctctatcac |
| | tgatagggagag. |

(SEQ ID NO: 44)

| | |
|---|---|
| 2O2_luc: | gatcaaagcttaaaaaaggacatcacttacgctgagt |
| | ctcttgaactcagcgtaagtgatgtccggtctctatc |
| | actgatagggag. |

(SEQ ID NO: 45)

| | |
|---|---|
| 2O2_HIF1A: | gatcaaagcttaaaaaagacagtacaggatgcttgct |
| | ctcttgaagcaagcatcctgtactgtcggtctctatc |
| | actgatagggag. |

e. Transcriptional Activity and Tetracycline Response of U6 Promoter Variants

The plasmids that use each of the U6 promoter variants to express shRNAs are designated as U6_luc, O1_luc, O1O2_luc1, O1O2_luc2, O1O2_luc3, O1O2_luc4, O1O2_luc5, and O1O2_luc6. Each of these plasmids (0.008 μg) or a control vector (the control vector is identical to the pU6 vector but does not contain a shRNA against luciferase) was co-transfected with 1 μg pGL3-control and 0.5 μg pRL-TK (The pGL3-control and pRL-TK plasmids each express firefly luciferase and renilla luciferase. The shRNA in each of these constructs are designed to inhibit firefly luciferase. The renilla luciferase was used for normalization purposes.). Transfection was carried out using Lipofectamine2000 (Invitrogen, Carlsbad, Calif.), according to the manufacturer's suggested protocol.

Luciferase activity in transfected cells was determined 72 hours post transfection. U6_luc, O1_luc, O1O2_{—}3, O1O2_{—}4, O1O2_luc5, O1O2_luc6 (0.2 μg each) and a control plasmid also were co-transfected separately with 1 μg pGL3-control, 0.5 μg pRL-TK and 1 μg of pcDNA6/TR. In addition, a control plasmid, U6_luc, O1_luc, O2_luc, and 2O2_luc (0.2 μg each) were co-transfected separately with 1 μg pGL3-control, 0.5 μg pRL-TK and 1 μg of pcDNA6/TR. For doxycycline treatment, cells were changed to culture medium containing 1 μg/ml of doxycycline 24 hours post transfection. Luciferase activity was determined 48 hours after induction by doxycycline.

f. Comparison of the tetO1 and 2O2 Expression Systems in Making Stable Cell Lines Expressing Luciferase shRNA

D54MG-tetR cells with stably integrated O1_luc (O1_luc1 . . . O1_luc4), 2O2_luc (2O2_luc1 . . . 2O2_luc7) or the 2O2 vector (control) were transfected with 1 μg pGL3-control and 0.5 μg pRL-TK. For doxycycline treatment, cells were changed into medium containing 1 μg/ml doxycycline 24 hours post transfection. Luciferase activities were determined 48 hours after induction by doxycycline. The cells were lysed after treatment with 1 μg/ml doxycycline for 48 hours and analyzed by western blotting using an anti-tetR antibody. The same blot was stripped and immunoblotted with an anti-actin antibody to show the equal loading of sample in each lane.

g. Comparison of the tetO1 and 2O2 Expression System in Making Stable Cell Lines Expressing Hif-1 shRNA

D54-MG-TetR cell lines with integrated O1_Hif1 cassette or 2O2_Hif1 cassette were treated with 1 μg/ml doxycycline for 48 hours followed by a six-hour treatment with 100 μM desferrioxamine (DFO). Cells were lysed in 1× Laemmli sample buffer and analyzed by western blotting using an antibody against Hif-1 alpha (1:500).

h. Results

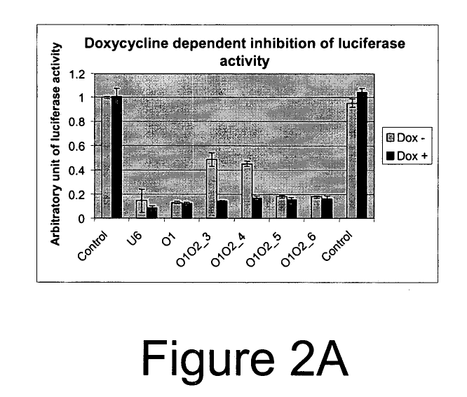

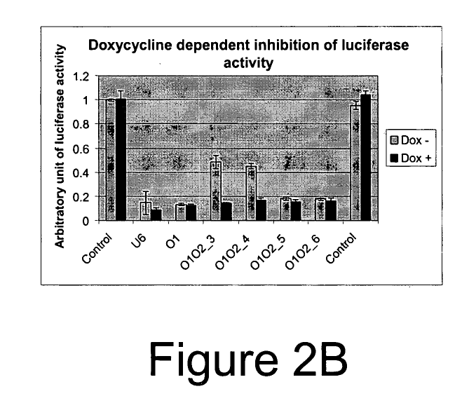

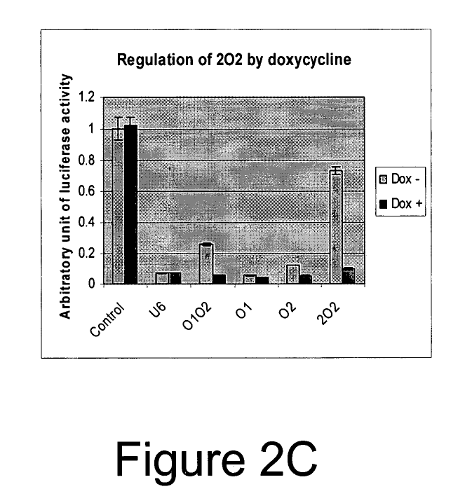

The inventors of the present invention first examined whether two tet operators can be engineered into the U6 promoter without abolishing the transcriptional activity. An O1 type tet operator was first engineered between the PSE and the TATA box to create a O1 type U6 promoter that is identical to that reported in Ohkawa, J., et al., Human Gene Therapy, 11:577-585 (2000) (See, FIG. 1, O1). A panel of modified human U6 promoters with two tet operators were then created by replacing part of the O1 type promoter with an O2 type tet operator (See FIG. 1). The transcriptional activities of the modified human U6 promoters were assessed by the ability of each promoter to express an shRNA targeting luciferase and inhibit the reporter activity. Based on a dose-response experiment using U6_luc, which utilizes the wild type U6 promoter to drive the expression of a luciferase shRNA, an amount of shRNA plasmid (0.008 μg) that exhibited 80% inhibition of the reporter activity was chosen for evaluation of the transcriptional activity exhibited by the modified U6 promoters. The degree of inhibition varied in cells transfected with U6 derivatives that contain both the O1 and O2 type tet operators. A similar degree of inhibition on luciferase activity was observed in cells transfected with O1_luc, O1O2_luc3, O1O2_luc4, O1O2_luc5, and O1O2_luc6, suggesting that introducing an additional O2 type tet operator into the O1 type promoter at these positions have only a marginal effect on the transcriptional activity (FIG. 2A, O1O2_{—}3, O1O2_{—}4, O1O2_{—}5, and O1O2_{—}6).

The active U6 promoter derivatives were then examined for their response to the inducing agent, doxycycline. Strong inhibition of luciferase activity was observed in cells transfected with O1_luc, O1O2_luc5, and O1O2_luc6 regardless of the presence or absence of doxycycline, suggesting that these promoters are very leaky under these experimental conditions (See, FIG. 2B, O1, O1O2_luc5, and O1O2_luc6). In contrast, cells transfected with O1O2_luc3 and O1O2_luc4 exhibited much lower luciferase activity in the presence of doxycyclin than in the absence of doxycycline. However, even in the absence of doxycycline, O1O2_luc3 and O1O2_luc4 transfected cells exhibited a >50% reduction of luciferase activity compared with cells transfected with a control vector (See, FIG. 2B, O1O2_{—}3, and O1O2_{—}4), suggesting that these promoters are still quite leaky despite of improved regulation compared to the O1 type promoter.

To further improve the inducible system, the O2 type tet operator was introduced to replace the O1 type tet operator in O1O2_{—}3 to generate a 2O2 type promoter (See, FIG. 1, 2O2). Because the O2 type tet operator has higher binding affinity for tetR than the O1 type tet operator (See, Hillen, W., et al., Annu. Rev. Microbiol., 48:345-69 (1994)), the inventors believed that it was likely that tetR would bind more tightly to the 2O2 type promoter than the O1O2_{—}3 type promoter, resulting in reduced basal transcriptional activity of the promoter. In the absence of doxycycline, O1O2_luc3 caused >70% reduction of the luciferase activity as compared with the control plasmid. Under the same condition, 2O2_luc caused no more than 30% inhibition of the luciferase activity (See, FIG. 2C), indicating that the 2O2 promoter indeed has less basal activity compared with the O1O2_{—}3 promoter. Meanwhile, O2_luc caused about 85% reduction of the luciferase activity, suggesting that two O2 type tet operators are needed at the same time to provide tight control of shRNA expression in the absence of doxycycline (See, FIG. 2C). In the presence of doxycycline, both O1O2_luc3 and 2O2_luc exhibited more than 80% inhibition of the luciferase activity, suggesting that the 2O2 and O1O2_{—}3 type promoters have similar activities upon induction (See, FIG. 2C). These results demonstrated that it is possible to engineering two tet operators into the U6 promoter without dramatically sacrificing the transcriptional activity. Meanwhile, with two O2 type tet operators flanking the TATA box, the resulting U6 promoter variant, 2O2, exhibited the best doxycycline response compared with U6 promoter variants with a single tet operator (O1 or O2) or a combination of O1 and O2 type tet operators.

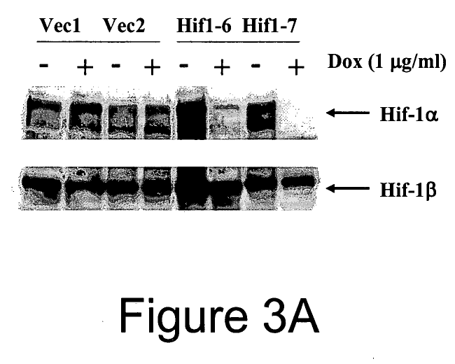

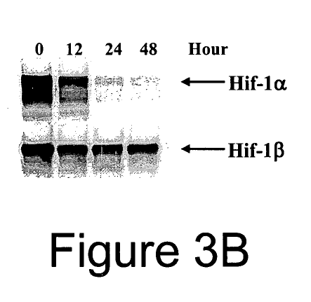

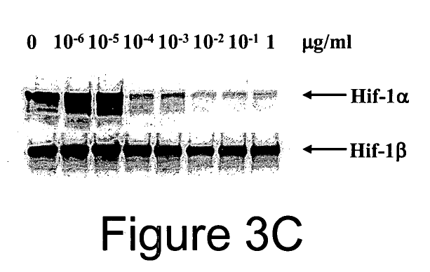

To determine whether the 2O2 promoter retains the ability to respond to doxycycline after integrating into chromosomes, the inventors used a commercial tetR expressing cell line, HeLaTREx, (Invitrogen Corp., Carlsbad, Calif. 92008) to establish stable clones that carried the 2O2 promoter linked to an shRNA targeting human Hif1α (2O2_Hif1). Among the five clones that carried the 2O2_Hif1 cassette, two clones exhibited a more than 90% reduction of HIF1α protein upon induction (See, FIG. 3A, Hif1-6 and Hif1-7). These results demonstrated that the 2O2 promoter retains its doxycycline responsive property after integrating into a chromosome.

Using the best-regulated 2O2_Hif1 clone (Hif1-7), the inventors further characterized the time and dose dependency of doxycycline induction of the 2O2 expression system. A significant reduction of Hif1α protein was observed as early as 12 hours after induction, and more than 90% inhibition of Hif-1 protein was observed 24 hours after doxycycline treatment. Longer induction did not lead to more complete inhibition of Hif1α protein (See, FIG. 3B). The doxycycline concentration that is required for maximal induction of the 2O2 system was determined in a dose-response experiment. A more than 90% inhibition of Hif-1 protein was observed in the presence of 0.1 ng/ml of doxycycline and the maximal inhibition of Hif1α protein was reached in the presence of 10 ng/ml of doxycycline (See, FIG. 3C). These results highlight the fast response and extreme sensitivity of the 2O2 system to doxycycline induction.

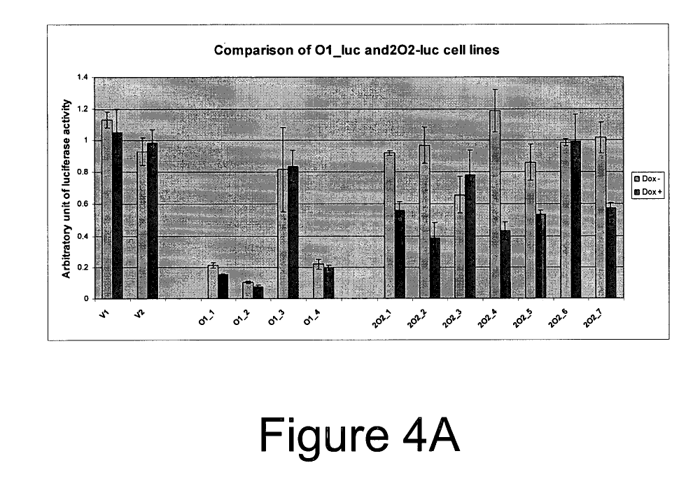

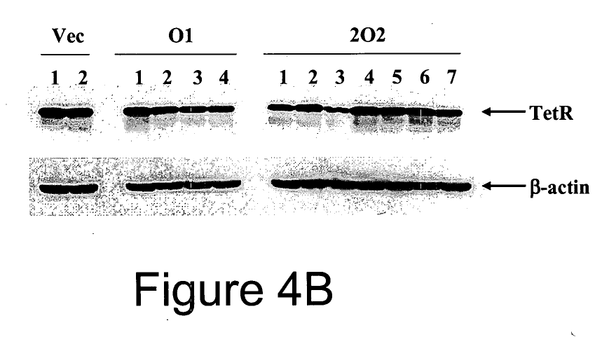

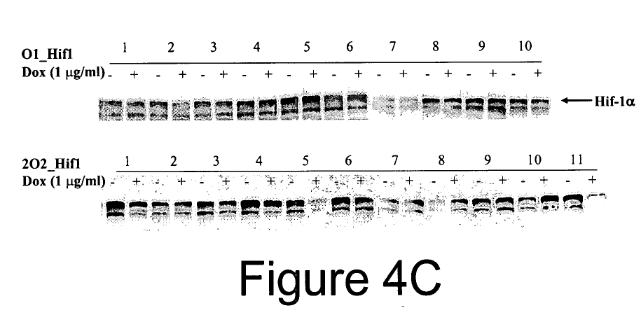

The use of pol III dependent inducible expression systems for regulated target knockdown is known in the art (See, van de Wetering, M., et al., EMBO Rep., 4:609-615 (2003), Matskura, S., et al., Nucleic Acid Res., 31:e77 (2003) and Czauderna, F., et al., Nucleic Acids Res., 31:e127 (2003)). In contrast to these reported observations, the inventors of the present invention observed severe leakiness of the O1 and O2 promoter in their initial studies (See, FIG. 2C, O1 and O2). To determine whether the observed leakiness of the system in the literature would have a negative impact on the ability of using these systems to create stable cell lines, the inventors directly compared the success rate of making inducible cell lines using both the O1 and the 2O2 systems. A D54MG cell line with high level of tetR expression was first established, and plasmids that utilizing the O1 or 2O2 promoters to drive the expression of shRNAs targeting luciferase (O1_luc and 2O2_luc) or human Hif1α (O1_Hif1 and 2O2_Hif1) were transfected with a hygromycine resistant gene into this cell line. The drug resistant clones were selected and analyzed by PCR to identify clones that carry the inducible shRNA expression cassette. The inventors obtained four clones with stably integrated O1_luc and seven clones with stably integrated 2O2_luc cassette as analyzed by PCR. All the clones displayed similar level of tetR expression (See, FIG. 4B). These clones were examined for their response to doxycycline induction. None of the four O1_luc clones exhibited significant doxycycline dependent reduction of luciferase activity (See, FIG. 4A, O1_luc1 to O1_luc4). Interestingly, three out of the four O1_luc clones exhibited constitutive inhibition of the luciferase activity regardless of the presence or absence of doxycycline, indicating severe leakiness of the O1 system (See, FIG. 4A, O1_luc1, O1_luc2, and O1_luc4). In contrast, among the seven 2O2_luc clones, two clones exhibited clear doxycycline dependent inhibition of luciferase activity (See, FIG. 4A, 2O2_luc2, 2O2_luc4), and three clones displayed modest degree of doxycycline dependent inhibition of luciferase activity (See, FIG. 4A, 2O2_luc 1, 2O2_luc5, and 2O2_luc7). The shRNA expression cassette for clone O1_{—}3, 2O2_{—}3 and 2O2_{—}6 could be inserted into transcriptional inactive site in a chromosome, resulting no inhibition of luciferase activity regardless of the presence or absence of doxycycline.

Similar results were also obtained from O1_Hif1 clones and 2O2_Hif1 clones. Among the ten O1_Hif1 clones analyzed, none of them exhibited apparent reduction of Hif1α protein upon doxycycline treatment (See, FIG. 4C, top). In contrast, two of the eleven 2O2_Hif1 clones exhibited significant reduction of Hif1α protein upon doxycycline treatment (See, FIG. 4C, bottom, clone 5 and 11).

### EXAMPLE 2

### Preparation of Cancer Cell Lines that Knockdown Hif1α Under the Control of Doxycycline

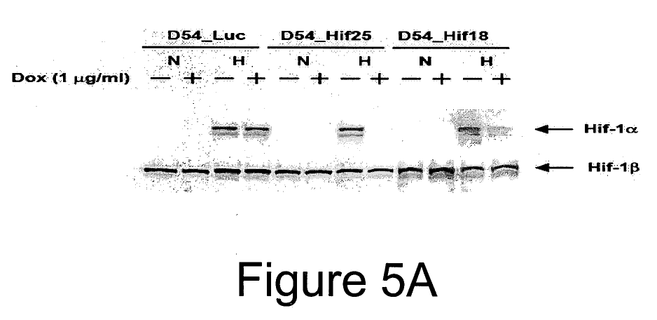

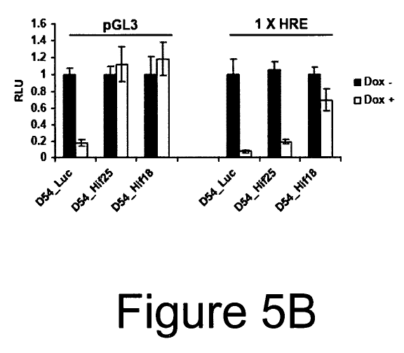

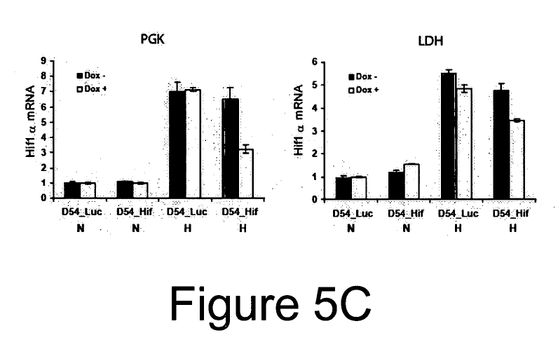

To assess the potential therapeutic effect of Hif1 inhibition, we established stable cell lines from D54MG parental cells that express an shRNA against Hif1α under the control of doxycycline. A panel of 8 shRNAs against Hif1α was first screened for their abilities to knockdown the target (data not shown), and the best shRNA was selected for the creation of Hif1α knockdown cell lines. The 2O2 promoter, a modified U6 promoter that tightly regulates the expression of shRNA (Xiaoyu Lin, In press), was chosen to drive the expression of the Hif1α siRNA. The stable clones that were produced exhibited variations in their ability to knockdown Hif1α upon doxycycline induction, presumably due to the effect of different integration sites on shRNA expression (FIG. 5A and data not shown). Compared with D54_Luc, a control cell line that expresses an shRNA against luciferase upon doxycycline induction, all D54_Hif clones produced similar levels of Hif1α protein in the absence of doxycycline, suggesting that the 2O2 expression system is very tightly regulated (FIG. 5A). Multiple clones that exhibited varying degrees of Hif1α knockdown after induction were further analyzed for Hif1 dependent transcriptional activity using a Hif1 reporter, which contains the HRE from the enolase promoter. Surprisingly, an 80% reduction of the Hif1α protein only had a marginal effect on the reporter activity (FIG. 5B, D54_Hif18). The lack of inhibition on the reporter activity was not due to the redundant function of Hif2α, because these cells have barely detectable Hif2α, and transfecting these cells with a potent siRNA against Hif2α failed to generate further inhibition of the reporter activity (data not shown). These results suggest that even very low levels of the Hif1α protein are sufficient to activate the transcription of Hif1-dependent downstream factors, and in the case of Hif1α, very high levels of knockdown are required in order to inhibit the Hif1 pathway.

In cells that exhibited complete knockdown of the Hif1α protein, an 80% reduction of the reporter activity was observed with doxycycline treatment (FIG. 5B, D54_Hif25). Transfecting these cells with a potent siRNA against Hif2α did not result in further inhibition of the reporter activity (data not shown), indicating that Hif1α is the predominant form in these cells. QPCR analysis indicated that doxycycline treatment in D54_Hif25 cells resulted in decreased transcription of Hif1 downstream factors such as PGK1, and LDH (FIG. 5C, D54_Hif25). As a control, the D54_Luc cells were also analyzed in parallel. Doxycycline treatment in D54_Luc cells caused the reduction of the luciferase reporter (FIG. 5B, D54_Luc) but not the knockdown of Hif1α protein (FIG. 5A, D54_Luc) or inhibition of Hif1 target genes (FIG. 5C, D54_Luc). These results demonstrated that expression of an shRNA against Hif1α resulted in the impairment of Hif1 dependent transcription, and this inhibitory effect resulted from the specific inhibition of Hif1α by shRNA rather than a non-specific effect due to the shRNA expression or doxycycline treatment.

### EXAMPLE 3

### Doxycycline Dependent Inhibition of Hif1α in Xenograft Tumors

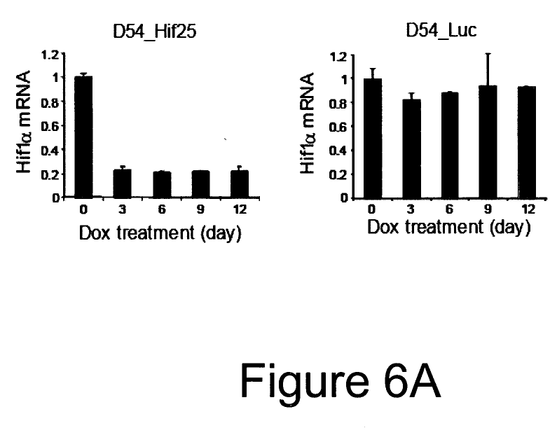

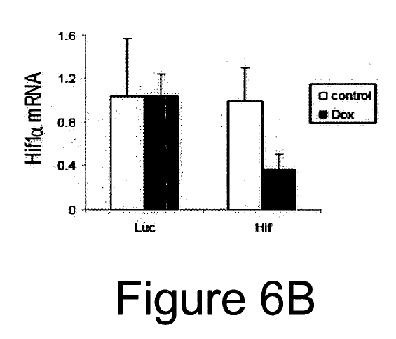

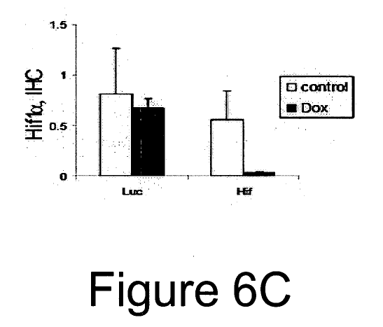

To determine whether target knockdown can be induced in xenograft tumors, the D54_Hif25 cells were injected subcutaneously into SCID mice. After tumors reached an average size of 200 mm³, the mice were supplied with drinking water containing 1 mg/ml doxycycline to induce the expression of Hif1α shRNA. After treating the mice with doxycycline for 3, 6, 9, or 12 days, the tumors were collected and analyzed by QPCR to determine the Hif1α messenger level. An 80% reduction of the Hif1α mRNA was observed in tumors from mice that received doxycycline for 3 days, and the knockdown was sustained over the entire 12-day treatment period (FIG. 6A). Examination of the tumor samples by immunohistochemistry indicated a clear reduction of the Hif1α protein from day 3 onward (data not shown).

To determine whether the ability to induce target knockdown would be impaired over long-term doxycycline treatment or when the tumors reach a very large size, further analysis was performed of the knockdown of Hif1α in tumors from mice treated with doxycycline for 45 days. The average tumor size at the end of treatment was 2000 mm³. A comparable degree of Hif1α knockdown was observed in these tumors compared with the tumors from mice that were under doxycycline treatment for 3 days (FIG. 6B). Immunohistochemistry analysis also demonstrated that the Hif1α protein was reduced to a barely detectable level in these tumors (FIG. 6C)). These results indicate that strong suppression of target expression can be sustained for a long period of time, even when tumors reached a large size. As a control, the D54_Luc cells were also injected subcutaneously to create xenograft tumors, and Hif1α knockdown was also examined in these tumors. No reduction of Hif1α mRNA or protein was observed in the D54_Luc tumors after doxycycline induction (FIGS. 6A, 6B, and 6C), suggesting that the reduction of Hif1α in the D54-Hif25 tumors result specifically from the expression of the Hif1α shRNA.

### EXAMPLE 4

### Effect of siRNA Mediated Inhibition of Hif1α on D54MG Tumor Growth

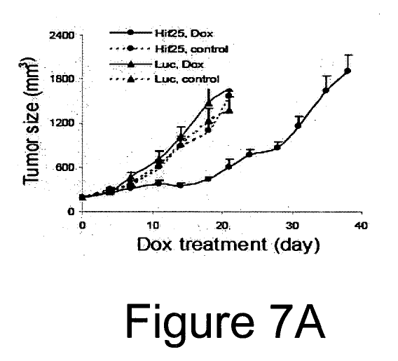

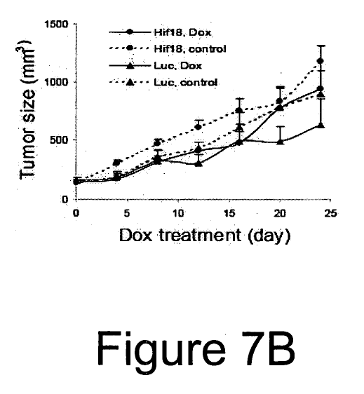

To assess the potential therapeutic effect of inhibiting Hif1α in established tumors, D54_Hif25 or D54_Luc (Luc) cells were used to generate xenograft tumors, and Hif1α knockdown was started when the tumors reached an average size of 190 mm³. Hif1α knockdown resulted in two phases of tumor growth. In the initial phase (FIG. 3A, day 1-day 11), tumors continued to grow but at a slightly slower growth rate compared to tumor with functional Hif1α. In the second phase, tumors exhibited a small but reproducible transient regression, then resumed growth without Hif1α (FIG. 5A, day 11 and afterwards). Although overall tumor growth was slower in the doxycycline treated group compared to the control group, all tumors in the treated group eventually grew to a large size, which resulted in the termination of the animals (FIG. 7A). Xenograft tumors generated from D54_Luc cells grew at the same rate regardless of the presence or absence of doxycycline in the drinking water, indicating that the slower growth phenotype of tumors expressing the Hif1α shRNA is a consequence of Hif1α knockdown (FIG. 7A). A similar inhibitory effect of Hif1α knockdown on tumor growth was observed using two independent clones that express Hif1α shRNA upon doxycycline treatment, demonstrating that the observed effect is consistent and not due to an aberrant clone (data not shown). These results suggest that the loss of Hif1α in established tumors caused a transient crisis that leads to tumor regression. However, tumors are able to adapt to the loss of Hif1α and continue to grow at a slower rate.

The availability of several clones that exhibited 80% knockdown of the Hif1α protein upon doxycycline treatment in vitro allowed for the determination of whether a partial inhibition of Hif1α will be sufficient to generate a therapeutic benefit in vivo. Xenograft tumors were generated using the D54_Hif18 cells, and Hif1α knockdown was initiated at an average tumor size of 150 mm³. Consistent with the lack of significant inhibition of Hif1-dependent transcription in vitro (FIG. 5B, D54_Hif18), doxycycline treatment failed to generate a significant effect on tumor growth in these tumors (FIG. 7B). These results suggest that a high degree of inhibition at the Hif1α protein level is required to negatively impact tumor growth in vivo.

### EXAMPLE 5

### Creation of Tyrosinase Knockdown Mice

a. Creation of Knockdown Mice Using Pronuclear Injection

Pronuclear injection is a well-established method for creating transgenic animals. In this approach, a DNA fragment (the transgene) is injected into the pronuclear stage of fertilized eggs, and the injected eggs are implanted into pseudopregnant animals. In a typical experiment, 50-80 eggs are injected in which half of the injected eggs will survive to generate neonates and 5%-20% of the neonates will contain the transgene.

Tyrosinase was selected as a target to knockdown. Tyrosinase is a key enzyme in melanin production, and the knockdown of tyrosinase in mice will generate an apparent coat color change.

b. Using Transgenes Driven by the 2O2 Promoter and Pronuclear Injection to Create Tyrosinase Knockdown Mice

To determine whether a modified pol III dependent promoter, such as the 2O2 promoter, is suitable for the creation of knockdown animals, transgenes were created that used the 2O2 promoters to express the two best shRNAs against tyrosinase (2O2-Tyr731 (SEQ ID NO: 46) and 2O2-Tyr338 (SEQ ID NO: 47)). These transgenes are shown below. The bold characters represent the shRNA sequences and the underscored characters represent the promoter sequences. The first set of injections were performed using embryos from mice that do not express the tet repressor (tetR). In mice without tetR expression, the 2O2 promoter is expected to be constitutively active.

Transgenes Driven by the 2O2 Promoter:

(SEQ ID NO: 46)

2O2-Tyr731:
gaattccaggcaaaacgcaccacgtgacggagcgtgaccgcgcgccgagc
gcgcgccaaggtcgggcaggaagagggcctatttcccatgattccatcat
atttgcatatacgatacaaggctgttagagagataattagaattaattcg
actgtaaacacaaagatattagtataaaatacgtgacgtagaaagtaata
atttcttgggtagtttgcagttttaaaattatgttttaaaatggactatc
atatgcttaccgtaacttgaaactccctatcagtgatagagattatatat
ctccctatcagtgatagagaccgtgacatttgcacagatgattcaagaga
tcatctgtgcaaatgtcacttttttaagctt

(SEQ ID NO: 47)

2O2-Tyr338:
gaattccaggcaaaacgcaccacgtgacggagcgtgaccgcgcgccgagc
gcgcgccaaggtcgggcaggaagagggcctatttcccatgattccatcat
atttgcatatacgatacaaggctgttagagagataattagaattaattcg
actgtaaacacaaagatattagtataaaatacgtgacgtagaaagtaata
atttcttgggtagtttgcagttttaaaattatgttttaaaatggactatc
atatgcttaccgtaacttgaaactccctatcagtgatagagattatatat
ctccctatcagtgatagagaccggcaacttcatgggtttcattcaagaga
tgaaacccatgaagttgccttttttaagctt

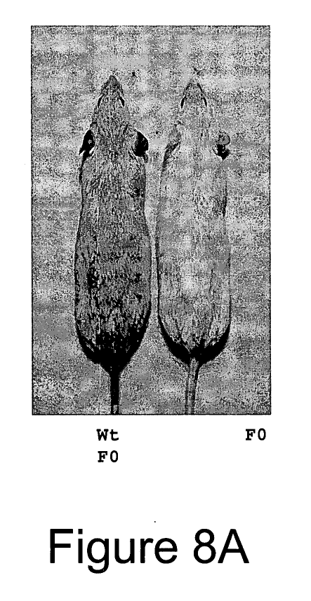

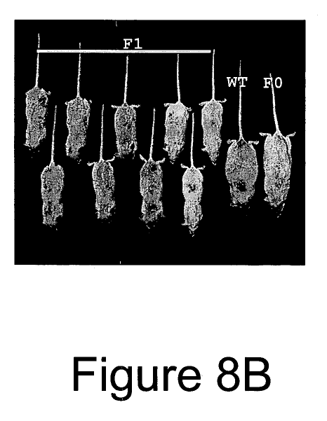

Among 20 pups born from embryos injected with the 2O2-Tyr731 transgene (SEQ ID NO: 46), three pups had a stably integrated transgene and exhibited different degrees of coat color change as shown in FIGS. 8A-B. FIG. 8A shows a lighter coat color of one F0 of the founders when compared to the darker coat color of the wild type mouse. The genotype of the mice was determined by PCR using transgene specific primers. FIG. 8B shows that the three pups are white in color compared to the darker color of the other F1 pups and are positive for the 2O2-Tyr731 transgene (SEQ ID NO: 46). These F1 transgenic progeny from the positive founders which exhibited a light coat color demonstrates that the siRNA mediated silencing effect can be transmitted through generations.

The embryos that were injected with the 2O2-Tyr338 transgene (SEQ ID NO: 47) gave rise to 40 pups and two dead embryos. Although none of the 40 neonates had the transgene, both dead embryos possessed the 2O2-Tyr338 transgene (SEQ ID NO: 47), suggesting that the 2O2-Tyr338 transgene (SEQ ID NO: 47) causes embryonic lethality due to the off-target effect of the Tyr338 shRNA.

These results suggest that the 2O2 promoter is well suited for the creation of knockdown animals.

Two parallel approaches for making conditional knockdown animals using the 2O2 system can be employed. The first approach involves the co-delivery of a 2O2-shRNA cassette with the CAGGS-tetR cassette in one transgene. Chicken beta actin promoter is a well-characterized promoter for ubiquitous gene expression. The CAGGS-tetR cassette utilizes the chicken beta actin promoter to drive the expression of tetR, which will result in the expression of tetR in the majority of mouse tissues. This approach can be used to create conditional knockdown animals in a short period of time.

The second approach involves the creation of a mouse line with ubiquitous tetR expression. This mouse line can then be used as the parental line for all conditional knockdown projects to achieve more uniform regulation of a target. It has been shown that genes at the ROSA26 locus are ubiquitously expressed. Therefore, a tetR expression cassette will be knocked in at the ROSA26 locus to obtain a mouse line with ubiquitous tetR expression.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

What is claimed is:

1. A RNA pol III dependent promoter sequence comprising a TATA element, a proximal sequence element (PSE) 5′ to the TATA element, and a transcriptional start site (TSS) 3′ to the TATA element, a first tetracycline operator located between the PSE and TATA element and a second tetracycline operator located between the TATA element and the TSS, wherein the first tetracycline operator has a polynucleotide sequence that is identical to a polynucleotide sequence of the second tetracycline operator.

2. The promoter sequence of claim 1 wherein the first tetracycline operator and the second tetracycline operator each have a polynucleotide sequence selected from the group consisting of: actctatcattgatagagttat (SEQ ID NO: 1), tccctatcagtgatagaga (SEQ ID NO:2), tccctatcagtgatagagacc (SEQ ID NO:3) and tccctatcagtgatagagagg (SEQ ID NO:4).

3. The promoter sequence of claim 1 wherein the promoter is a U6 promoter, H1 promoter or a 7SK promoter.

4. A RNA pol III dependent promoter sequence comprising a TATA element, a proximal sequence element (PSE) 5′ to the TATA element, and a transcriptional start site (TSS) 3′ to the TATA element, a first tetracycline operator located between the PSE and TATA element and which forms a portion of the PSE or TATA element and a second tetracycline operator located between the TATA element and the TSS, wherein the first tetracycline operator has a polynucleotide sequence that is identical to a polynucleotide sequence of the second tetracycline operator.

5. The promoter sequence of claim 4 wherein the first tetracycline operator and the second tetracycline operator each have a polynucleotide sequence selected from the group consisting of: actctatcattgatagagttat (SEQ ID NO: 1), tccctatcagtgatagaga (SEQ ID NO:2), tccctatcagtgatagagacc (SEQ ID NO:3) and tccctatcagtgatagagagg (SEQ ID NO:4).

6. The promoter sequence of claim 5 wherein the promoter is a U6 promoter, H1 promoter or a 7SK promoter.

7. A RNA pol III dependent promoter sequence comprising a TATA element, a proximal sequence element (PSE) 5′ to the TATA element, and a transcriptional start site (TSS) 3′ to the TATA element, a first tetracycline operator located between the PSE and TATA element and a second tetracycline operator located between the TATA element and the TSS, wherein the first tetracycline operator has a polynucleotide sequence that is different than a polynucleotide sequence of the a second tetracycline operator, provided that when the first tetracycline operator has the polynucleotide sequence of: actctatcattgatagagttat (SEQ ID NO: 1), the second tetracycline operator does not have a polynucleotide sequence of: ctccctatcagtgatagagaaa (SEQ ID NO:5).

8. The promoter sequence of claim 7 wherein the second tetracycline operator has a polynucleotide sequence selected from the group consisting of: tccctatcagtgatagaga (SEQ ID NO:2), tccctatcagtgatagagacc (SEQ ID NO:3) and tccctatcagtgatagagagg (SEQ ID NO:4).

9. The promoter sequence of claim 7 wherein the first tetracycline operator has the polynucleotide sequence of: tccctatcagtgatagagacc (SEQ ID NO:2) and the second tetracycline operator has the polynucleotide sequence of: actctatcattgatagagttat (SEQ ID NO: 1).

10. The promoter sequence of claim 7 wherein the promoter is a U6 promoter, H1 promoter or a 7SK promoter.

11. A RNA pol III dependent promoter sequence comprising a TATA element, a proximal sequence element (PSE) 5′ to the TATA element, and a transcriptional start site (TSS) 3′ to the TATA element, a first tetracycline operator located between the PSE and TATA element and which forms a portion of the PSE or TATA element and a second tetracycline operator located between the TATA element and the TSS, wherein the first tetracycline operator has a polynucleotide sequence that is different than a polynucleotide sequence of the second tetracycline operator, provided that when first tetracycline operator has the polynucleotide sequence of: actctatcattgatagagttat (SEQ ID NO: 1), the second tetracycline operator has a polynucleotide sequence of: ctccctatcagtgatagagaaa (SEQ ID NO: 5).

12. The promoter sequence of claim 11 wherein the second tetracycline operator has a polynucleotide sequence selected from the group consisting of: tccctatcagtgatagaga (SEQ ID NO:2), tccctatcagtgatagagacc (SEQ ID NO:3) and tccctatcagtgatagagagg (SEQ ID NO:4).

13. The promoter sequence of claim 11 wherein the first tetracycline operator has the polynucleotide sequence of: tccctatcagtgatagagacc (SEQ ID NO:2) and the second tetracycline operator has the polynucleotide sequence of: actctatcattgatagagttat (SEQ ID NO: 1).

14. The promoter sequence of claim 11 wherein the promoter is a U6 promoter, H1 promoter or a 7SK promoter.

15. A vector comprising:
at least one RNA pol III dependent promoter sequence of claim 1 operably linked to at least one polynucleotide sequence of interest.

16. The vector of claim 15 wherein the at least one polynucleotide sequence of interest is DNA or cDNA.

17. A vector comprising:
at least one RNA pol III dependent promoter sequence of claim 5 operably linked to at least one polynucleotide sequence of interest.

18. The vector of claim 17 wherein the at least one polynucleotide sequence of interest is DNA or cDNA.

19. A vector comprising:
at least one RNA pol III dependent promoter sequence of claim 7 operably linked to at least one polynucleotide sequence of interest.

20. The vector of claim 19 wherein at least one polynucleotide sequence of interest is DNA or cDNA.

21. A vector comprising:
at least one RNA pol III dependent promoter sequence of claim 11 operably linked to at least one polynucleotide sequence of interest.

22. The vector of claim 21 wherein the at least one polynucleotide sequence of interest is DNA or cDNA.

23. An eukaryotic cell comprising the vector of claim 15.

24. An eukaryotic cell comprising the vector of claim 17.

25. An eukaryotic cell comprising the vector of claim 19.

26. An eukaryotic cell comprising the vector of claim 21.

27. A transgenic non-human animal comprising: a transgene comprising at least one polynucleotide sequence of interest operably linked to a RNA pol III dependent promoter sequence, wherein transcription of said polynucleotide sequence of interest produces an RNA molecule that modulates expression of at least one target gene in said transgenic non-human animal and further wherein said promoter sequence comprises a TATA element, a proximal sequence element (PSE) 5′ to the TATA element, and a transcriptional start site (TSS) 3′ to the TATA element, a first tetracycline operator located between the PSE and TATA element and a second tetracycline operator located between the TATA element and the TSS, wherein the first tetracycline operator has a polynucleotide sequence that is identical to a polynucleotide sequence of the second tetracycline operator.

28. The animal of claim 27 wherein the first tetracycline operator and the at second tetracycline operator each have a polynucleotide sequence selected from the group consisting of: actctatcattgatagagttat (SEQ ID NO: 1), tccctatcagtgatagaga (SEQ ID NO:2), tccctatcagtgatagagacc (SEQ ID NO:3) and tccctatcagtgatagagagg (SEQ ID NO:4).

29. The animal of claim 27 wherein the promoter is a U6 promoter, H1 promoter or a 7SK promoter.

30. The animal of claim 27, wherein said animal is selected from the group consisting of: mouse rat, dog, cat, pig, cow, goat, sheep, primate and guinea pig.

31. The animal of claim 27 wherein at least one polynucleotide sequence of interest is DNA or cDNA.

32. The animal of claim 27 wherein the RNA molecule is small interferring RNA or short hairpin RNA.


SEQUENCE LISTING (47 sequences)

SEQ ID NO: 1 - DNA, 22 bp, Artificial Sequence
  Tetracycline operator
actctatcattgatagagttat

SEQ ID NO: 2 - DNA, 19 bp, Artificial Sequence
  Polynucleotide
tccctatcagtgatagaga

SEQ ID NO: 3 - DNA, 21 bp, Artificial Sequence
  Polynucleotide
tccctatcagtgatagagacc

SEQ ID NO: 4 - DNA, 21 bp, Artificial Sequence
  Polynucleotide
tccctatcagtgatagagagg

SEQ ID NO: 5 - DNA, 22 bp, Artificial Sequence
  Tetracycline O2 type U6 promoter
ctccctatcagtgatagagaaa

SEQ ID NO: 6 - DNA, 93 bp, Artificial Sequence
  U6 promoter variants
ttaaaatggactatcatatgtcatatgcttaccgtaacttgaaagtatttcgatttcttggctttatatatcttgtggaaaggacgaaacacc

SEQ ID NO: 7 - DNA, 94 bp, Artificial Sequence
  U6 promoter variants
ttaaaatggactatcatatgtcatatgcttaccgtaacttgaaagtactctatcattgatagagttatatatcttgtggaaaggacgaaacacc

SEQ ID NO: 8 - DNA, 93 bp, Artificial Sequence
  U6 promoter variants
ttaaaatggactatcatatgtcatatgcttaccgtaacttgaaagtatttcgatttcttggctttatatatctccctatcagtgatagagacc

SEQ ID NO: 9 - DNA, 94 bp, Artificial Sequence
  U6 promoter variants
ttaaaatggactatcatatgtcatatgcttaccgtaacttgaaagtactctatcattgatagagttatatatccctatcagtgatagagaaacc

SEQ ID NO: 10 - DNA, 94 bp, Artificial Sequence
  U6 promoter variants
ttaaaatggactatcatatgtcatatgcttaccgtaacttgaaagtactctatcattgatagagttatatatccctatcagtgatagagacacc

SEQ ID NO: 11 - DNA, 94 bp, Artificial Sequence
  U6 promoter variants
ttaaaatggactatcatatgtcatatgcttaccgtaacttgaaagtactctatcattgatagagttatatatctccctatcagtgatagagacc

SEQ ID NO: 12 - DNA, 94 bp, Artificial Sequence
  U6 promoter variants
ttaaaatggactatcatatgtcatatgcttaccgtaacttgaaagtactctatcattgatagagttatatatctctccctatcagtgatagaga

SEQ ID NO: 13 - DNA, 94 bp, Artificial Sequence
  U6 promoter variants
ttaaactccctatcagtgatagagatgcttaccgtaacttgaaagtactctatcattgatagagttatatatcttgtggaaaggacgaaacacc

SEQ ID NO: 14 - DNA, 94 bp, Artificial Sequence
  U6 promoter variants
ctccctatcagtgatagagatcatatgcttaccgtaacttgaaagtactctatcattgatagagttatatatcttgtggaaaggacgaaacacc

SEQ ID NO: 15 - DNA, 94 bp, Artificial Sequence
  U6 promoter variants
ttaaaatggactatcatatgtcatatgcttaccgtaacttgaaactccctatcagtgatagagattatatatctccctatcagtgatagagacc

SEQ ID NO: 16 - RNA, 49 nt, Artificial Sequence
  Chemically synthesized short hairpin RNA
ggaagaaauuagcaguagcucucuugaagcuacugcuaauuucuuccuu

SEQ ID NO: 17 - DNA, 21 bp, Artificial Sequence
  variation 20, 21: n = T/U,  Combined DNA/RNA molecule; chemically synthesized small interfering  RNA (siRNA)
gggcauuucucagagaaugnn

SEQ ID NO: 18 - DNA, 21 bp, Artificial Sequence
  variation 1, 2: n = T/U, Combined DNA/RNA molecule; chemically synthesized small interfering  RNA (siRNA)
nncccguaaagagucucuuac

SEQ ID NO: 19 - DNA, 75 bp, Artificial Sequence
  PCR primer
gatcgaattccaggcaaaacgcaccacgtgacggagcgtgaccgcgcgccgagcgcgcgccaaggtcgggcagga

SEQ ID NO: 20 - DNA, 75 bp, Artificial Sequence
  PCR primer
aacagccttgtatcgtatatgcaaatatgatggaatcatgggaaataggccctcttcctgcccgaccttggcgcg

SEQ ID NO: 21 - DNA, 75 bp, Artificial Sequence
  PCR primer
atatacgatacaaggctgttagagagataattagaattaatttgactgtaaacacaaagatattagtataaaata

SEQ ID NO: 22 - DNA, 75 bp, Artificial Sequence
  PCR primer
aaacataattttaaaactgcaaactacccaagaaattattactttctacgtcacgtattttatactaatatcttt

SEQ ID NO: 23 - DNA, 75 bp, Artificial Sequence
  PCR primer
gcagttttaaaattatgttttaaaatggactatcatatgcttaccgtaacttgaaagtatttcgatttcttggct

SEQ ID NO: 24 - DNA, 61 bp, Artificial Sequence
  PCR primer
tctagaagcttggtgtttcgtcctttccacaagatatataaagccaagaaatcgaaatact

SEQ ID NO: 25 - DNA, 30 bp, Artificial Sequence
  PCR primer
gatcgaattccaggcaaaacgcaccacgtg

SEQ ID NO: 26 - DNA, 30 bp, Artificial Sequence
  PCR primer
tctagaagcttggtgtttcgtcctttccac

SEQ ID NO: 27 - DNA, 76 bp, Artificial Sequence
  PCR primer
ggtgtttcgtcctttccacaagatatataactctatcaatgatagagtactttcaagttacggtaagcatatgata

SEQ ID NO: 28 - DNA, 49 bp, Artificial Sequence
  PCR primer
tttctctatcactgatagggagatatataaagccaagaaatcgaaatac

SEQ ID NO: 29 - DNA, 57 bp, Artificial Sequence
  PCR primer
tctagaagcttggtgtttcgtcctttccacaagatatataactctatcaatgataga

SEQ ID NO: 30 - DNA, 44 bp, Artificial Sequence
  PCR primer
ggtttctctatcactgatagggatatataactctatcaatgata

SEQ ID NO: 31 - DNA, 56 bp, Artificial Sequence
  PCR primer
ggtgtctctatcactgatagggatatataactctatcaatgatagagtactttcaa

SEQ ID NO: 32 - DNA, 46 bp, Artificial Sequence
  PCR primer
ggtctctatcactgatagggagatatataactctatcaatgataga

SEQ ID NO: 33 - DNA, 48 bp, Artificial Sequence
  PCR primer
tctctatcactgatagggagagatatataactctatcaatgatagagt

SEQ ID NO: 34 - DNA, 85 bp, Artificial Sequence
  PCR primer
ataactctatcaatgatagagtactttcaagttacggtaagcatctctatcactgatagggaacataattttaaaactgcaaact

SEQ ID NO: 35 - DNA, 87 bp, Artificial Sequence
  PCR primer
ataactctatcaatgatagagtactttcaagttacggtaagcatatgatctctatcactgatagggaattttaaaactgcaaactac

SEQ ID NO: 36 - DNA, 80 bp, Artificial Sequence
  PCR primer
ggtctctatcactgatagggagatatataatctctatcactgatagggagtttcaagttacggtaagcatatgatagtcc

SEQ ID NO: 37 - DNA, 85 bp, Artificial Sequence
  PCR primer
gatcaaagcttaaaaaaggacatcacttacgctgagtctcttgaactcagcgtaagtgatgtccggtgtttcgtcctttccacaa

SEQ ID NO: 38 - DNA, 79 bp, Artificial Sequence
  PCR primer
tagaagcttaaaaaggacatcacttacgctgagtctcttgaactcagcgtaagtgatgtcctttctctatcactgatag

SEQ ID NO: 39 - DNA, 85 bp, Artificial Sequence; identical to SEQ ID NO: 37
  PCR primer

SEQ ID NO: 40 - DNA, 86 bp, Artificial Sequence
  PCR primer
gatcaaagcttaaaaaaggacatcacttacgctgagtctcttgaactcagcgtaagtgatgtccggtttctctatcactgataggg

SEQ ID NO: 41 - DNA, 86 bp, Artificial Sequence
  PCR primer
gatcaaagcttaaaaaaggacatcacttacgctgagtctcttgaactcagcgtaagtgatgtccggtgtctctatcactgataggg

SEQ ID NO: 42 - DNA, 86 bp, Artificial Sequence
  PCR primer
gatcaaagcttaaaaaaggacatcacttacgctgagtctcttgaactcagcgtaagtgatgtccggtctctatcactgatagggag

SEQ ID NO: 43 - DNA, 86 bp, Artificial Sequence
  PCR primer
gatcaaagcttaaaaaaggacatcacttacgctgagtctcttgaactcagcgtaagtgatgtcctctctatcactgatagggagag

SEQ ID NO: 44 - DNA, 86 bp, Artificial Sequence; identical to SEQ ID NO: 42
  PCR primer

SEQ ID NO: 45 - DNA, 86 bp, Artificial Sequence
  PCR primer
gatcaaagcttaaaaaagacagtacaggatgcttgctctcttgaagcaagcatcctgtactgtcggtctctatcactgatagggag

SEQ ID NO: 46 - DNA, 381 bp, Artificial Sequence
  2O2-Tyr731
gaattccaggcaaaacgcaccacgtgacggagcgtgaccgcgcgccgagcgcgcgccaaggtcgggcaggaagagggcctatttcccatgattccatcatatttgcatatacgatacaaggctgttagagagataattagaattaattcgactgtaaacacaaagatattagtataaaatacgtgacgtagaaagtaataatttcttgggtagtttgcagttttaaaattatgttttaaaatggactatcatatgcttaccgtaacttgaaactccctatcagtgatagagattatatatctccctatcagtgatagagaccgtgacatttgcacagatgattcaagagatcatctgtgcaaatgtcacttttttaagctt

SEQ ID NO: 47 - DNA, 381 bp, Artificial Sequence
  2O2-Tyr338
gaattccaggcaaaacgcaccacgtgacggagcgtgaccgcgcgccgagcgcgcgccaaggtcgggcaggaagagggcctatttcccatgattccatcatatttgcatatacgatacaaggctgttagagagataattagaattaattcgactgtaaacacaaagatattagtataaaatacgtgacgtagaaagtaataatttcttgggtagtttgcagttttaaaattatgttttaaaatggactatcatatgcttaccgtaacttgaaactccctatcagtgatagagattatatatctccctatcagtgatagagaccggcaacttcatgggtttcattcaagagatgaaacccatgaagttgccttttttaagctt